(19) United States
(12) Patent Application Publication
(10) Pub. No.: US 2017/0282184 A1
(43) Pub. Date: Oct. 5, 2017

(54) THERMAL CYCLER LID CONFIGURATIONS AND USES THEREOF

(71) Applicant: Coyote Bioscience Co., Ltd., Beijing (CN)

(72) Inventors: Xiang LI, Beijing (CN); Yuguang HAN, Chengde (CN); Xiaobing MU, Beijing (CN)

(21) Appl. No.: 15/356,382

(22) Filed: Nov. 18, 2016

Related U.S. Application Data
(63) Continuation of application No. PCT/CN2015/079500, filed on May 21, 2015.
Application No. PCT/CN2015/079500 is a continuation-in-part of application No. PCT/CN2014/078026, filed on May 21, 2014.

(51) Int. Cl. B01L 7/00; C12Q 1/68; F27D 11/00; C12M 1/38

(52) U.S. Cl. CPC B01L 7/52; C12M 1/38; C12Q 1/68; F27D 11/00; B01L 2300/043; B01L 2300/041; B01L 2300/046; B01L 2300/1805

(57) ABSTRACT

Lid configurations for thermal cyclers and uses thereof are provided. A nucleic acid amplification device may include a lid assembly that may be able to move between an open and closed configuration. The lid assembly may include a heater therein. The heater in the lid may have an adjustable configuration that may be able to accommodate different configurations of sample containers. Optionally, the heater may be retractable within a housing of the thermal cycler, when the lid is opened.

### CROSS-REFERENCE

The present application is a continuation of PCT Patent Application Serial No. PCT/CN2015/079500 filed on May 21, 2015, which is a continuation-in-part of PCT Patent Application Serial No. PCT/CN2014/078026 filed on May 21, 2014, each of which is incorporated herein by reference in its entirety.

### BACKGROUND OF THE INVENTION

Nucleic acid amplification methods permit selected amplification and identification of nucleic acids of interest from a complex mixture, such as a biological sample. Nucleic acid of interest can be amplified via amplification methods known in the art, such as thermal cycling based approaches including polymerase chain reaction (PCR). During or following amplification of the nucleic acid of interest, the products of amplification can be detected and results of the detection interpreted by an end user.

Lids may be provided for a thermal cycling device that may be capable of performing nucleic acid amplification. The lids may be used to contain a sample within the thermal cycling device.

### SUMMARY OF THE INVENTION

A need exists for improved lid configurations for thermal cyclers. Improved lid configurations may aid in rapid and effective thermal cycling of samples within the thermal cycler. For example, the lids may include heated portions that may contact sample containers within which the samples may reside and aid in controlling the temperature of the sample and/or permitting detection of the samples within the containers. The lids may have features that may accommodate different sample containers having different shapes or dimensions. The lids may also permit a user to open the lid and add or remove sample while reducing the risk of the user contacting the heated portions. These features can greatly improve the ability to perform thermal cycling in different settings, and make the loading and unloading of the thermal cycler simplified.

Aspects of the invention are directed to an apparatus for processing a sample within a device, said apparatus comprising: a lid configured to rotate about at least one axis of rotation when said lid is moving between an open position and a closed position; and a heater configured to translate along a plane without rotating when said lid is moving between the open position and the closed position, wherein said translation is driven by the rotation by the lid, and wherein said heater is configured to cover the sample when the lid is in the closed position, and wherein said heater is configured to not cover the sample when the lid is in the open position.

In some embodiments, the device is a thermal cycler. The thermal cycler may be useful for performing real-time nucleic acid amplification, and said thermal cycler comprises a detector configured to detect a signal from the sample while the nucleic acid amplification is in progress, wherein the signal is related to the amount of amplified nucleic acid present in the sample.

The sample may be contained within a sample container. The sample container may have a container top. The heater may be configured to contact the container top of the sample container when the lid is in the closed position.

The translation may be mechanically driven by the rotation of the lid. The lid may be coupled to a slider pin that is positioned along a track, wherein movement of the lid causes the slider pin to slide along the track. The lid may be coupled to the slider pin via a rigid connector that is capable of pivoting about an axis in relation to the slider pin. The rigid connector may be capable of pivoting about an axis in relation to the lid. The heater may be coupled to the slider pin and is configured to slide along with the slider pin.

In some implementations, the apparatus may further comprise a steel wire connected to the heater, wherein said translation of the heater causes the steel wire to change length. The apparatus may further comprise a guiding block for the steel wire. The steel wire may be configured to bend when the lid is in the closed position.

Additional aspects of the invention may be directed to a thermal cycler for performing nucleic acid amplification, said thermal cycler comprising: the apparatus as previously described; and a processor configured to generate instructions to the heater to control temperature of the heater.

The thermal cycler may further comprise a housing configured to enclose the sample and the processor. The lid may form a portion of the housing. The heater may be within the housing when the lid is in the open position.

Aspects of the invention may be directed to a method for controlling access to a sample, said method comprising: providing a device having the apparatus as previously described; and receiving an input to move the lid between the open position and the closed position. The input may be received manually by a user pressing a button on the device

An apparatus for processing a sample within a device may be provided in accordance with additional aspects of the invention. The apparatus may comprise: a lid configured to move between an open position and a closed position; and a heater configured to be separable from the lid and to move along a different path from the lid when said lid is moving between the open position and the closed position, wherein movement of said heater is mechanically driven by movement of the lid, and wherein said heater is configured to cover the sample when the lid is in the closed position, and wherein said heater is configured to not cover the sample when the lid is in the open position.

In some embodiments, device may be a thermal cycler. The thermal cycler may be useful for performing real-time nucleic acid amplification, and said thermal cycler comprises a detector configured to detect a signal from the sample while the nucleic acid amplification is in progress, wherein the signal is related to the amount of amplified nucleic acid present in the sample.

The sample may be contained within a sample container. The sample container may have a container top. The heater may be configured to contact the container top of the sample container when the lid is in the closed position.

The lid may be coupled to a slider pin that is positioned along a track, wherein movement of the lid causes the slider pin to slide along the track. The lid may be coupled to the slider pin via a rigid connector that is capable of pivoting about an axis in relation to the slider pin. The rigid connector may be capable of pivoting about an axis in relation to the lid. The heater may be coupled to the slider pin and is configured to slide along with the slider pin.

The apparatus may further comprise a steel wire connected to the heater, wherein said movement of the heater causes the steel wire to change length. The apparatus may further comprise a guiding block for the steel wire. The steel wire may be configured to bend when the lid is in the closed position.

Further aspects of the invention may be directed to a thermal cycler for performing nucleic acid amplification, said thermal cycler comprising: the apparatus as previously described; and a processor configured to generate instructions to the heater to control temperature of the heater.

The thermal cycler may further comprise a housing configured to enclose the sample and the processor. The lid may form a portion of the housing. The heater may be within the housing when the lid is in the open position.

Additionally, aspects of the invention may include a method for controlling access to a sample, said method comprising: providing a device having the apparatus as previously described; and receiving an input to move the lid between the open position and the closed position. The input may be received manually by a user pressing a button on the device

In accordance with further aspects of the invention, an apparatus for processing a sample within a device may be provided. The apparatus may comprise: a lid configured to move between an open position and a closed position; and a heater configured to move when said lid is moving between the open position and the closed position, and wherein said heater is configured to (1) cover the sample and (2) contact the lid via a force inducing component that exerts a force on the heater toward the sample when the lid is in the closed position, and wherein said heater is configured to (1) not cover the sample and (2) not contact the lid via the force inducing component when the lid is in the open position.

The heater may be configured to move along a different path from the lid. The movement of said heater may be mechanically driven by movement of the lid. The force inducing component may be a spring. The spring may be a leaf spring.

The heater may be configured to move along a path that is different from a path of the lid between the open position and the closed position.

In some embodiments, the device may be a thermal cycler. The thermal cycler may be useful for performing real-time nucleic acid amplification, and said thermal cycler comprises a detector configured to detect a signal from the sample while the nucleic acid amplification is in progress, wherein the signal is related to the amount of amplified nucleic acid present in the sample.

The sample may be contained within a sample container. The sample container may have a container top. The heater may be configured to contact the container top of the sample container when the lid is in the closed position.

Aspects of the invention may be further directed to a thermal cycler for performing nucleic acid amplification, said thermal cycler comprising: the apparatus as previously described; and a processor configured to generate instructions to the heater to control temperature of the heater.

The thermal cycler may further comprise a housing configured to enclose the sample and the processor. The lid may form a portion of the housing. The heater may be within the housing when the lid is in the open position.

Additional aspects of the invention may be directed to a method for controlling access to a sample, said method comprising: providing a device having the apparatus as previously described; and receiving an input to move the lid between the open position and the closed position. The input may be received manually by a user pressing a button on the device.

An aspect of the invention may be directed to a thermal cycler for performing nucleic acid amplification in a sample, said thermal cycler comprising: a sample holder configured to support one or more sample containers containing the sample therein; and a lid configured to move between an open position and a closed position, wherein said lid includes a heater coupled to a force inducing component that causes the heater to contact and exert force on the one or more sample containers when the lid is in the closed position, when the one or more sample containers have a height falling between 15 mm and 22 mm.

The heater may be configured to contact and exert force on the one or more sample containers when the sample containers are 0.2 mL tubes having a flat cap, and when the sample containers are 0.2 mL tubes having a domed cap. The heater may be capable of moving relative to an outer surface of the lid, wherein a range of the movement is at least 4 mm. The heater may be configured to remain parallel relative to an outer surface of the lid during the movement.

The thermal cycler may further comprise a processor configured to generate instructions to the heater to control temperature of the heater.

In some embodiments, the force inducing component may be one or more coil springs. The force inducing component may be one or more leaf springs.

The thermal cycler may be useful for performing real-time nucleic acid amplification and monitoring, and said thermal cycler further comprises a detector configured to detect a signal from the sample while the nucleic acid amplification is in progress.

The thermal cycler may further comprise a switch, wherein sliding the switch mechanically may cause a lid to move from a closed position to the open position. The lid may move from a closed position to the open position by rotating about an axis. The lid may rotate about the axis with aid of a torsion spring that exerts a force on the lid to move toward the open position. Moving the lid from an open position to a closed position may cause the lid to remain closed without further manual interference. The switch may be coupled to a buckle that is configured to slide over a portion of the lid to keep it closed and slide away from the portion of the lid to permit the lid to open. The buckle may be coupled to a spring that induces a force on the buckle to keep it over the portion of the lid until a user slides the switch to overcome the force exerted by the spring.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows an example of a device for conducting a nucleic acid amplification reaction in accordance with an embodiment of the invention.

FIG. 1B shows another example of a device for conducting a nucleic acid amplification reaction in accordance with an embodiment of the invention.

FIG. 2 shows samples that may be provided within sample containers supported by a device in accordance with an embodiment of the invention.

FIG. 3 shows an example of a thermal cycle in accordance with an embodiment of the invention.

FIG. 4 shows an example of a device and a display in accordance with an embodiment of the invention.

FIG. 5 shows an example of power being provided to a device that includes an energy storage device in accordance with an embodiment of the invention.

FIG. 6 shows an example of a device for conducting a nucleic acid amplification reaction in accordance with an embodiment of the invention.

FIG. 7 shows an example of a lid configuration for a device in accordance with an embodiment of the invention.

FIG. 8 shows an additional example of a lid configuration for a device in accordance with an embodiment of the invention.

FIG. 9 shows an exterior view of a lid and instrument panel for a device in accordance with an embodiment of the invention.

FIG. 10 provides an additional view of a lid configuration for a device in accordance with an embodiment of the invention.

FIG. 11 shows a view of a lid in an open position in accordance with an embodiment of the invention.

FIG. 12 shows an additional view of a lid in an open position in accordance with an embodiment of the invention.

FIG. 13 shows a close up view of a lid release mechanism in accordance with an embodiment of the invention.

FIG. 14 shows a cross-sectional view of a lid configuration with adjustable portion in accordance with an embodiment of the invention.

FIG. 15 shows a cross-sectional view of a lid configuration with adjustable portion in accordance with an embodiment of the invention.

FIG. 16 shows multiple views of a device having a lid in accordance with an embodiment of the invention.

FIG. 17 shows a view of a device with a lid in a partially opened position in accordance with an embodiment of the invention.

FIG. 18 shows a view of a device with a lid in an open position in accordance with an embodiment of the invention.

FIG. 19 shows views of a device with a lid being closed in accordance with an embodiment of the invention.

FIG. 20 shows a device with a lid in an open position with a retractable portion in accordance with an embodiment of the invention.

FIG. 21 shows a lid apparatus in a partially opened position in accordance with an embodiment of the invention.

FIG. 22 shows a lid apparatus in an open position in accordance with an embodiment of the invention.

FIG. 23 shows a lid apparatus in a closed position in accordance with an embodiment of the invention.

FIG. 24 shows a locking mechanism for a lid apparatus in accordance with an embodiment of the invention.

FIG. 25 shows a lid apparatus where the locking mechanism has been released in accordance with an embodiment of the invention.

FIG. 26 shows a lid apparatus being opened in accordance with an embodiment of the invention.

FIG. 27 shows a lid apparatus being closed in accordance with an embodiment of the invention.

FIG. 28 shows an additional view of a lid apparatus being opened in accordance with an embodiment of the invention.

FIG. 29 shows an additional view of a lid apparatus in an open position in accordance with an embodiment of the invention.

FIG. 30 shows an additional view of a lid apparatus being closed in accordance with an embodiment of the invention.

FIG. 31 shows an example of dimensions within which a device for conducting nucleic acid amplification may fall, in accordance with an embodiment of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides lid configurations for thermal cycler devices and uses thereof. Various aspects of the invention described herein may be applied to any of the particular applications set forth below or for any other types of nucleic acid amplification systems. The invention may be applied as a standalone system or method, or as part of an integrated sample processing system. It shall be understood that different aspects of the invention can be appreciated individually, collectively, or in combination with each other.

A thermal cycler may contain one or more samples therein. The samples may be provided within sample containers that may be supported by a supporting element within the thermal cycler. The supporting element may be a heating and/or cooling block. The supporting element may be used to control the temperature of the sample within the thermal cycler. Optionally, a detector may be provided to detect the samples. The detector may detect a signal from the samples, such as an optical signal. The thermal cycler may have a housing enclosing one or more elements of the thermal cycler.

The thermal cycler may have a lid. The lid may be provided on a housing of the thermal cycler. The lid may open or close. When the lid is opened, a sample container may be inserted into the thermal cycler or removed from the thermal cycler. When the lid is closed, access to the sample containers and/or supporting element may be blocked by the lid. The lid may include a heating element. The heating element of the lid may be configured to contact the sample container(s) supported by the supporting element when the lid is closed. The heating element of the lid may aid in temperature control of the sample within the sample container. Optionally, the heating element may also be useful for permitting a detector to detect the sample within the sample container. For example, the heating element may control the temperature of a container top and prevent condensation or fogging of the container top. This may provide a detector with a clearer view of the sample within the container.

The lid may have an adjustable component that may permit the thermal cycler to accommodate sample containers of different configurations. For example, the lid may have a heated adjustable component that may permit both dome topped and flat topped sample containers to be accepted within the thermal cycler. The heated adjustable component may contact the top of the various types of sample containers and may be able to adjust for different heights of tops of the sample containers. This may advantageously permit the same thermal cycler to accommodate different sample holders. The lid configuration may provide increased flexibility to the user of the thermal cycler.

A heated portion of the lid may move with the lid. For example, if the lid is opened and/or closed by pivoting about an axis, the heated portion may move with the lid. In another embodiment, the heated portion of the lid may be separable from the outer surface of the lid. For example, if an outer part of the lid is opened and closed by pivoting about an axis, the heated portion may be retractable and/or slide under a portion of a housing of the thermal cycler. The heated portion may be able to move without rotating. The motion of heated portion may be driven by the movement of the outer portion of the lid. In some embodiments, the heated portion may move in a manner that may prevent a user from contacting the heated portion when the lid is open. This may advantageously reduce the risk of burns or injuries to the user.

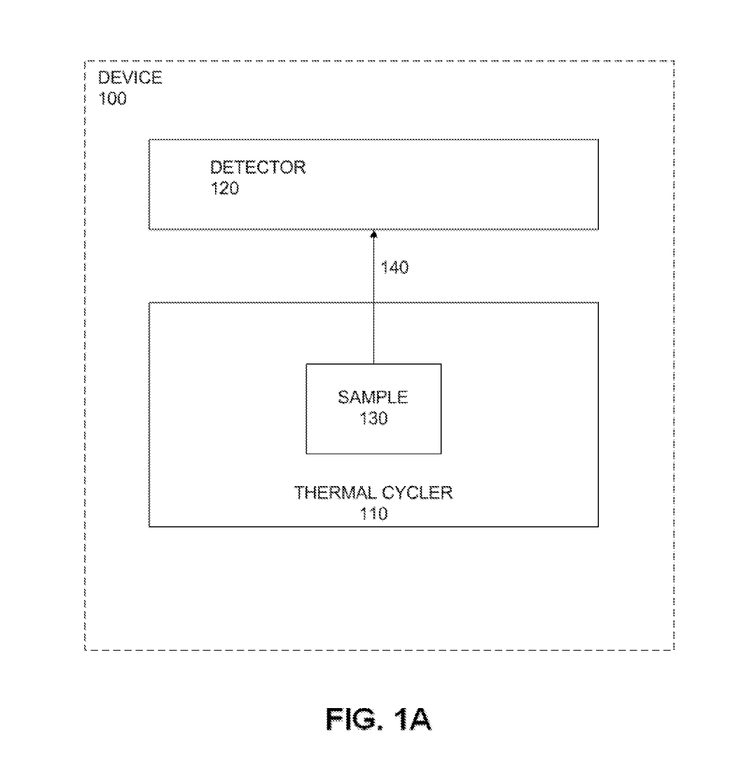

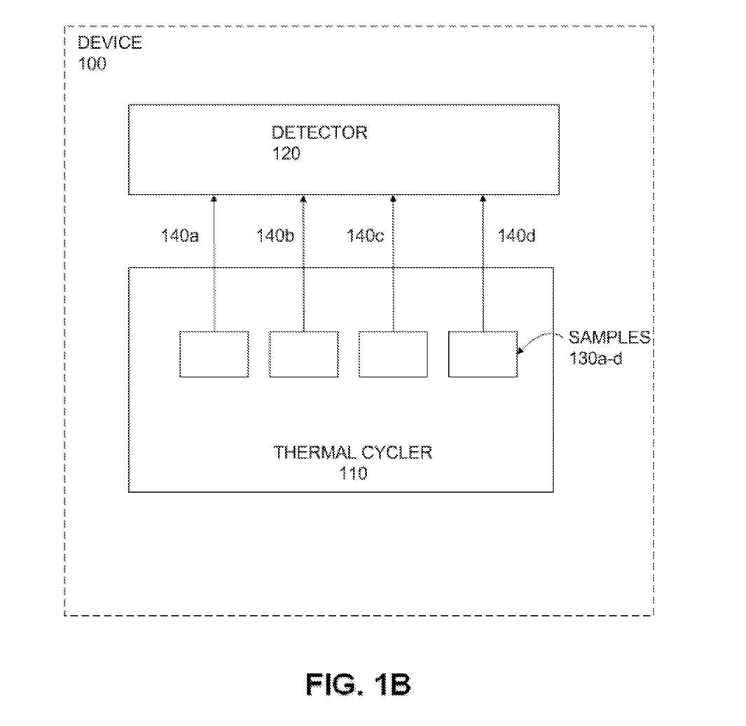

FIG. 1A shows an example of a device for conducting a nucleic acid amplification reaction in accordance with an embodiment of the invention. A device 100 for conducting nucleic acid amplification may include a thermal cycler 110 and, optionally, a detector 120. The thermal cycler may be configured to accept a sample 130.

The thermal cycler 110 may be capable of receiving the sample 130 which may comprise a target nucleic acid. The sample may also include an agent that detects amplified target nucleic acid (e.g., a detectable nucleic acid binding agent). The sample may also include other reagents for conducting a nucleic acid amplification. Depending on the nature of the target nucleic acid that is to be amplified, other reagents may include reverse transcriptase for conducting reverse-transcriptase coupled PCT, dNTPs, Mg²⁺ ion.

The sample 130 may be a biological sample. The biological sample may be taken from a subject. For example, the sample may be taken from a living subject directly. In some embodiments, the biological sample may include breath, blood, urine, feces, saliva, cerebrospinal fluid and sweat. Any suitable biological sample that comprises nucleic acid may be obtained from a subject. A biological sample may be solid matter (e.g., biological tissue) or may be a fluid (e.g., a biological fluid). In general, a biological fluid can include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

A subject may be a living subject or a dead subject. The subject may be a human or an animal. In some examples, the subject may be mammal. Examples of subjects may include, but are not limited to simians, avines, canines, felines, equines, bovines, ovines, porcines, delphines, rodents (e.g., mice, rats), or insects.

A biological sample may be obtained from a subject by any means known in the art. Non-limiting examples of means to obtain a biological sample directly from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab), pipetting, and breathing. Moreover, a biological sample may be obtained from any anatomical part of a subject where a desired biological sample is located.

A biological sample obtained directly from a subject may generally refer to a biological sample that has not been further processed after being obtained from the subject, with the exception of any means used to collect the biological sample from the subject for further processing. For example, blood is obtained directly from a subject by accessing the subject's circulatory system, removing the blood from the subject (e.g., via a needle), and entering the removed blood into a receptacle. The receptacle may comprise reagents (e.g., anti-coagulants) such that the blood sample is useful for further analysis. In another example, a swab may be used to access epithelial cells on an oropharyngeal surface of the subject. After obtaining the biological sample from the subject, the swab containing the biological sample can be contacted with a fluid (e.g., a buffer) to collect the biological fluid from the swab. Alternatively, pre-processing may occur on the biological sample prior to being provided to the device.

In some embodiments, a biological sample has not been purified when provided in a reaction vessel. In some embodiments, the nucleic acid of a biological sample has not been extracted when the biological sample is provided to a reaction vessel. For example, the RNA or DNA in a biological sample may not be extracted from the biological sample when providing the biological sample to a reaction vessel. Moreover, in some embodiments, a target nucleic acid (e.g., a target RNA or target DNA) present in a biological sample may not be concentrated prior to providing the biological sample to a reaction vessel. Alternatively, dilution or concentration of the sample may occur prior to being provided to a device.

The sample 130 may have a target nucleic acid to be amplified. The target nucleic acid may be amplified to generate an amplified product. A target nucleic acid may be a target RNA or a target DNA. In cases where the target nucleic acid is a target RNA, the target RNA may be any type of RNA. In some embodiments, the target RNA is viral RNA. In some embodiments, the viral RNA may be pathogenic to the subject. Non-limiting examples of pathogenic viral RNA include human immunodeficiency virus I (HIV I), human immunodeficiency virus II (HIV II), orthomyxoviruses, influenza viruses (e.g., H1N1, H3N2, H5N1), hepevirus, hepatitis A, hepatitis B, hepatitis C, hepatitis D, hepatitis E, hepatitis G, Epstein-Barr virus, mononucleosis, cytomegalovirus, SARS, West Nile Fever, polio, and measles.

In cases where the target nucleic acid is a target DNA, the target DNA may be any type of DNA. In some embodiments, the target DNA is viral DNA. In some embodiments, the viral DNA may be pathogenic to the subject. Non-limiting examples of DNA viruses include herpes simplex virus, smallpox, and chickenpox. In some cases, a target DNA may be a bacterial DNA. The bacterial DNA may be from a bacterium pathogenic to the subject such as, for example, Mycobacterium tuberculosis—a bacterium known to cause tuberculosis.

The sample 130 may also include an agent that detects amplified target nucleic acid. The agent may be a reporter agent that can yield a detectable signal whose presence or absence is indicative of the presence of an amplified product. The intensity of the detectable signal may be proportional to the amount of amplified product. For example, the detectable signal may be directly linearly proportional, exponentially proportional, reversely proportional, or have any other type of proportional relationship to the amount of amplified product. In some cases, where amplified product is generated of a different type of nucleic acid than the target nucleic acid initially amplified, the intensity of the detectable signal may be proportional to the amount of target nucleic acid initially amplified. For example, in the case of amplifying a target RNA via parallel reverse transcription and amplification of the DNA obtained from reverse transcription, reagents necessary for both reactions may also comprise a reporter agent may yield a detectable signal that is indicative of the presence of the amplified DNA product and/or the target RNA amplified. The intensity of the detectable signal may be proportional to the amount of the amplified DNA product and/or the original target RNA amplified. The use of a reporter agent also enables real-time amplification methods, including real-time PCR for DNA amplification.

Reporter agents may be linked with nucleic acids, including amplified products, by covalent or non-covalent means. Non-limiting examples of non-covalent means include ionic interactions, Van der Waals forces, hydrophobic interactions, hydrogen bonding, and combinations thereof. In some embodiments, reporter agents may bind to initial reactants and changes in reporter agent levels may be used to detect amplified product. In some embodiments, reporter agents may only be detectable (or non-detectable) as nucleic acid amplification progresses. In some embodiments, an optically-active dye (e.g., a fluorescent dye) may be used as may be used as a reporter agent. An agent for detecting amplified target nucleic acid may be a nucleic acid binding dye. The dye may be a DNA-intercalating dye. Non-limiting examples of dyes include Eva green, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5-(or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some instances, a reporter agent may be a sequence-specific oligonucleotide probe that can be optically active when hybridized with an amplified product. Due to sequence-specific binding of the probe to the amplified product, use of oligonucleotide probes can increase specificity and sensitivity of detection. A probe may be linked to any of the optically-active reporter agents (e.g., dyes) described herein and may also include a quencher capable of blocking the optical activity of an associated dye. Non-limiting examples of probes that may be useful used as reporter agents include TaqMan probes, TaqMan Tamara probes, TaqMan MGB probes, or Lion probes.

A reporter agent may be an RNA oliognucleotide probe that may include an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe with the exonuclease activity of a DNA polymerase during amplification, the quencher and dye are separated, and the free dye regains its optical activity that can subsequently be detected.

Optionally, a reporter agent may be a molecular beacon. A molecular beacon may include, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and can be detected. The sequence specificity of the molecular beacon for a target sequence on the amplified product can improve specificity and sensitivity of detection.

In some embodiments, a reporter agent may be a radioactive species. Non-limiting examples of radioactive species include ¹⁴C, ¹²³I, ¹²⁴I, ¹²⁵I, Tc99m, ³⁵S, or ³H.

In some embodiments, a reporter agent may be an enzyme that is capable of generating a detectable signal. Detectable signal may be produced by activity of the enzyme with its substrate or a particular substrate in the case the enzyme has multiple substrates. Non-limiting examples of enzymes that may be used as reporter agents include alkaline phosphatase, horseradish peroxidase, I²-galactosidase, alkaline phosphatase, β-galactosidase, acetylcholinesterase, and luciferase.

The sample 130 may be provided with reagents necessary for nucleic acid amplification within the device. In some instances, a reagent may comprise one or more of the following: (i) a reverse transcriptase, (ii) a DNA polymerase, and (iii) a primer set for the target nucleic acid (e.g., RNA). Some examples of reagents may include a commercially available pre-mixture (e.g., Qiagen One-Step RT-PCR or One-Step RT-qPCR kit) comprising reverse transcriptases (e.g., Sensiscript and Omniscript transcriptases), a DNA Polymerase (e.g., HotStarTaq DNA Polymerase), and dNTPs.

In some instances, the sample 130 may be provided within a sample container, such as a reaction vessel. Any components of the sample including the target nucleic acid, agent that detects amplified target nucleic acid, and/or reagents for nucleic acid amplification may be provided within the reaction vessel to obtain a reaction mixture. Any suitable reaction vessel may be used. In some embodiments, a reaction vessel comprises a body that can include an interior surface, an exterior surface, an open end, and an opposing closed end. In some embodiments, a reaction vessel may comprise a cap. The cap may be configured to contact the body at its open end, such that when contact is made the open end of the reaction vessel is closed. In some cases, the cap is permanently associated with the reaction vessel such that it remains attached to the reaction vessel in open and closed configurations. In some cases, the cap is removable, such that when the reaction vessel is open, the cap is separated from the reaction vessel. In some embodiments, a reaction vessel may be sealed, optionally hermetically sealed. The reaction vessel may be fluid-tight.

A reaction vessel may be of varied size, shape, weight, and configuration. In some examples, a reaction vessel may be round or oval tubular shaped. In some embodiments, a reaction vessel may be rectangular, square, diamond, circular, elliptical, or triangular shaped. A reaction vessel may be regularly shaped or irregularly shaped. In some embodiments, the closed end of a reaction vessel may have a tapered, rounded, or flat surface. For example, a flat cap, rounded cap, or tapered cap may be provided. The cap may be a dome cap. The cap surface may be flat, smooth, ridged, grooved, or may include bumps, protrusions, holes, indentations, or any other types of features. Non-limiting examples of types of a reaction vessel include a tube, a well, a capillary tube, a cartridge, a cuvette, a centrifuge tube, or a pipette tip.

Any dimensions may be provided for a reaction vessel. The reaction vessel may be configured to contain no more than 0.1 mL, 0.2 mL or 0.5 mL of sample. The reaction vessel may be configured to contain no more than about 0.01 mL, 0.03 mL, 0.05 mL, 0.07 mL, 0.1 mL, 0.12 mL, 0.15 mL, 0.17 mL, 0.2 mL, 0.22 mL, 0.25 mL, 0.27 mL, 0.3 mL, 0.32 mL, 0.35 mL, 0.37 mL, 0.4 mL, 0.42 mL, 0.45 mL, 0.47 mL, 0.5 mL, 0.52 mL, 0.55 mL, 0.6 mL, 0.7 mL, 0.8 mL, 0.9 mL, 1 mL, 1.1 mL, 1.2 mL, 1.3 mL, 1.5 mL, 1.7 mL, 2 mL, 2.5 mL, 3 mL, 3.5 mL, 4 mL, 5 mL, 6 mL, or 7 mL. The reaction vessel may be configured to contain more than any of the values described herein. The reaction vessel may have a volume configured to contain no more than a volume falling into a range between two of the values described herein.

The reaction vessel may be less than or equal to about 15 mm, 15.2 mm, 15.8 mm, 20.8 mm, 21.4 mm, 21.5 mm, 21.6 mm, 21.7 mm, 21.8 mm, 12.9 mm, or 22 mm tall. The reaction vessel may have a height of less than or equal to about 1 mm, 2 mm, 3 mm, 4 mm, 5 mm, 6 mm, 7 mm, 8 mm, 9 mm, 10 mm, 12 mm, 13 mm, 14 mm, 15 mm, 16 mm, 17 mm, 18 mm, 19 mm, 20 mm, 21 mm, 22 mm, 23 mm, 24 mm, 25 mm, 27 mm, 30 mm, 35 mm, 40 mm, 45 mm, 50 mm, 55 mm, 60 mm, or 70 mm. The reaction vessel may have a height greater than any of the values described herein. The reaction vessel may have a height falling into a range between any two of the values described herein.

The reaction vessel may have a cross-sectional area of no more than 0.001 mm², 0.005 mm², 0.01 mm², 0.03 mm², 0.05 mm², 0.1 mm², 0.12 mm², 0.15 mm², 0.2 mm², 0.3 mm², 0.4 mm², 0.5 mm², 0.6 mm², 0.7 mm², 0.8 mm², 0.9 mm², 1 mm², 1.1 mm², 1.2 mm², 1.3 mm², 1.5 mm², 1.7 mm², 2 mm², 2.2 mm², 2.5 mm², 3 mm², 3.5 mm², 4 mm², 4.5 mm², 5 mm², 6 mm², 7 mm², 8 mm², 9 mm², 10 mm², 12 mm², 15 mm², 17 mm², 20 mm², 22 mm², 25 mm², 30 mm², 35 mm², 40 mm², or 50 mm². The reaction vessel may have a cross-sectional area less than any of the values described herein. The reaction vessel may have a cross-sectional area falling into a range between any two of the values described herein.

Reaction vessels may be constructed of any suitable material with non-limiting examples of such materials that include glasses, metals, plastics, and combinations thereof. Reaction vessels can be made from optically transparent or translucent materials that may permit an optical signal from within the reaction vessel to leave the reaction vessel. The reaction vessels may be made from a material that may or may not filter an optical signal exiting the reaction vessel. In some instances, the reaction vessels may be formed from a clear material that may permit a detector to view the interior of the reaction vessels. In some instances, the interior of the reaction vessels may be imaged. Alternatively, an amount of optical signal exiting the reaction vessel may be detected and measured. In some instances, a cap or top surface of the reaction vessel may be opaque, translucent, or transparent. The cap or top surface of the reaction vessel may be clear. The cap and/or top surface of the vessel may permit an optical signal to exit the reaction vessel through the top. The optical signal exiting the top may or may not be filtered. The optical signal exiting the top may or may not be collimated, focused, or dispersed.

A thermal cycler may be capable of receiving a reaction vessel. The reaction vessels may be removably provided to the thermal cycler. The reaction vessels may be inserted within a device or taken out of the device. The reaction vessels may be placed onto a supporting component of the thermal cycler or taken off the supporting component.

In alternative embodiments, the sample may be loaded directly into the device without requiring a separate reaction vessel. In some instances, reaction vessels or receptacles may be directly built-into the device.

The thermal cycler 110 may accept the reaction vessel having the sample therein, or may directly receive the sample. The thermal cycler may be capable of alternatingly heating and cooling the sample. Multiple cycles of heating and cooling may be provided. Any temperature profile may be provided for the various heating and cooling cycles.

The thermal cycler may utilize conduction, convection, and/or radiation to heat and/or cool the samples. In one example, a heating block may be provided that may directly contact the sample, or may contact a sample container that contains the sample. The heating block may be capable of heating and/or cooling the sample. In some instances, electricity may be used to resistively heat a heating/cooling system of the thermal cycler. Other techniques, such as induction heating may be used to control the heating/cooling system of the thermal cycler. In some instances Peltier devices may be used to heat or cool the samples in the thermal cycler.

Any type of nucleic acid amplification reaction known in the art may be used to amplify a target nucleic acid and generate an amplified product. Moreover, amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction, ligase chain reaction, helicase-dependent amplification, asymmetric amplification, rolling circle amplification, and multiple displacement amplification (MDA). In some embodiments, the amplified product may be DNA. In cases where a target RNA is amplified, DNA can be obtained by reverse transcription of the RNA and subsequent amplification of the DNA can be used to generate an amplified DNA product. The amplified DNA product may be indicative of the presence of the target RNA in the biological sample. In cases where DNA is amplified, any DNA amplification method known in the art may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, DNA amplification is linear. In some cases, DNA amplification is exponential. In some cases, DNA amplification is achieved with nested PCR, which can improve sensitivity of detecting amplified DNA products.

Nucleic acid amplification reactions described herein may be conducted in parallel, in some implementations. Parallel amplification reactions may be amplification reactions that can occur in the same reaction vessel and at the same time. Parallel nucleic acid amplification reactions may be conducted, for example, by including reagents necessary for each nucleic acid amplification reaction in a reaction vessel to obtain a reaction mixture and subjecting the reaction mixture to conditions necessary for each nucleic amplification reaction. For example, reverse transcription amplification and DNA amplification may be conducted in parallel, by providing reagents necessary for both amplification methods in a reaction vessel to form to obtain a reaction mixture and subjecting the reaction mixture to conditions suitable for conducting both amplification reactions. DNA generated from reverse transcription of the RNA may be amplified in parallel to generate an amplified DNA product. Any suitable number of nucleic acid amplification reactions may be conducted in parallel. In some cases, at least 1, 2, 3, 4, 5, 6, 7, 8, 9, 10, 11, 12, 13, 14, 15, 16, 17, 18, 19, 20, or more nucleic acid amplification reactions are conducted in parallel.

Time may elapse while nucleic acid amplification reactions are occurring. A detector 120 of the device 100 may be capable of detecting a signal during the time while the nucleic acid amplification reaction is occurring. The detector may be capable of detecting the signal without removing the sample 130 from the device.

In various aspects, the detector 120 may detect amplified product (e.g., amplified DNA product, amplified RNA product). Detection of amplified product, including amplified DNA, may be accomplished with any suitable detection method known in the art. The particular type of detection method used may depend, for example, on the particular amplified product, the type of reaction vessel used for amplification, other reagents in a reaction mixture, whether or not a reporter agent was included in a reaction mixture, and if a reporter agent was used, the particular type of reporter agent use. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

The detector 120 may be capable of detecting an optical signal from the sample 130. The optical signal may be a fluorescent or other luminescent signal from the sample. The optical signal may be generated by the sample in response to a stimulation light provided to the sample. A stimulation light may be provided by a light source. The light source may be within the device 100. In some instances, light may be absorbed by the sample, and the sample may emit light. The emitted light may be at the same or different wavelength from the emitted light. In some instances, the optical signal may be a reflection of light from the light source. Alternatively, light may be shined through the sample, and the detector may be capable of detecting the light that passes through the sample.

In some embodiments, information regarding the presence of and/or an amount of amplified product (e.g., amplified DNA product) may be outputted to a recipient. Information regarding amplified product may be outputted via any suitable means known in the art. Such information may be provided in real-time while the nucleic-acid amplification is underway. In other instances, the information may be provided once the nucleic acid amplification has been completed. In some instances, some data may be provided in real-time while other data may be presented once the amplification is completed.

In some embodiments, such information may be provided verbally to a recipient. In some embodiments, such information may be provided in a report. A report may include any number of desired elements, with non-limiting examples that include information regarding the subject (e.g., sex, age, race, health status, etc.) raw data, processed data (e.g. graphical displays (e.g., figures, charts, data tables, data summaries), determined cycle threshold values, calculation of starting amount of target polynucleotide), conclusions about the presence of the target nucleic acid, diagnosis information, prognosis information, disease information, and the like, and combinations thereof. The report may be provided as a printed report (e.g., a hard copy) or may be provided as an electronic report. In some embodiments, including cases where an electronic report is provided, such information may be outputted via an electronic display, such as a monitor or television, a screen operatively linked with a unit used to obtain the amplified product, a tablet computer screen, a mobile device screen, and the like. Both printed and electronic reports may be stored in files or in databases, respectively, such that they are accessible for comparison with future reports.

Moreover, a report may be transmitted to the recipient at a local or remote location using any suitable communication medium including, for example, a network connection, a wireless connection, or an internet connection. In some embodiments, a report can be sent to a recipient's device, such as a personal computer, phone, tablet, or other device. The report may be viewed online, saved on the recipient's device, or printed. A report can also be transmitted by any other suitable means for transmitting information, with non-limiting examples that include mailing a hard-copy report for reception and/or for review by a recipient.

Moreover, such information may be outputted to various types of recipients. Non-limiting examples of such recipients include the subject from which the biological sample was obtained, a physician, a physician treating the subject, a clinical monitor for a clinical trial, a nurse, a researcher, a laboratory technician, a representative of a pharmaceutical company, a health care company, a biotechnology company, a hospital, a human aid organization, a health care manager, an electronic system (e.g., one or more computers and/or one or more computer servers storing, for example, a subject's medical records), a public health worker, other medical personnel, and other medical facilities.

The device 100 that may include the thermal cycler 110 and, optionally, the detector 120 may include a housing. The housing may partially or completely enclose components of the device. The housing may surround components of the device laterally and/or on the top and bottom. The housing may optionally be a rigid structure. For example, the housing may contain the thermal cycler therein. Optionally, the detector may also be contained within the housing. In other implementations, the detector may be outside the housing of the device. The detector may be an integral part of the device. Alternatively, the detector may be removable or separable from the device.

An optical path 140 may be provided between the sample 130 and the detector 120. A signal from the sample may reach the detector via the optical path. An optical signal from a sample may traverse the optical path to reach the detector. The optical path may include direct line-of-sight between the sample and the detector. In some instances, one or more optical elements may be provided between the sample and the detector. Examples of optical elements may include lenses, mirrors, prisms, diffusers, concentrators, filters, dichroics, optical fibers, or any other type of optical elements. The optical elements may be built into a sample container, such as a cap of the sample container. Alternatively or in addition, optical elements may be provided in the device, separately from the sample container. The optical elements may or may not be provided in a lid of a device, or other portion of the device.

Optionally, the optical path 140 may be provided entirely within a housing of the device 100. The housing may optically isolate the optical path from the surrounding environment. For example, the housing may be light-tight so that little or no interfering optical signals may be provided within the housing that may interfere with the optical path. Light from outside the housing may not be capable of entering the interior of the housing. This may advantageously reduce inaccuracies in the optical signal detected by the detector 120.

The optical path 140 may remain while the nucleic acid amplification is occurring. The detector may be able to continuously or periodically detect signals from the ample while the nucleic acid amplification is occurring via the optical path.

In some instances, the detector may be part of the device and may be used to detect signals from the sample in real-time. Real-time PCR may occur using the device.

In other instances, a detector need not be built into the thermal cycler. A detector may be external to the thermal cycler. In some instances, samples may be removed from the thermal cycler and may undergo detection. Optionally, detection does not occur while nucleic acid amplification is occurring. Detection may occur after the nucleic acid amplification and/or the thermal cycles have been completed. Thus, the PCR performed by the thermal cycler need not be real-time PCR. Any examples herein pertaining to detectors on-board the thermal cycler and real-time PCR may also apply to PCR that is not real-time, or thermal cyclers that are used for PCR that is not real-time PCR.

FIG. 1B shows another example of a device for conducting a nucleic acid amplification reaction in accordance with an embodiment of the invention. The device 100 may include a thermal cycler 110 and, optionally, a detector 120. The thermal cycler may be capable of receiving a plurality of samples 130a-d. Optical paths 140a-d may be provided between the samples and the detector.

In some embodiments, a plurality of samples 130a-d may be provided to the thermal cycler. The thermal cycler may be capable of receiving a plurality of samples. The thermal cycler may be capable of receiving the number of samples loaded therein, or may be capable of receiving more than the samples loaded therein. The thermal cycler may have sites capable of receiving samples, and the sites may or may not all be filled. For example, the thermal cycler may be capable of receiving 8 samples, but may have 400 or fewer, 300 or fewer, 200 or fewer, 100 or fewer, 75 or fewer, 50 or fewer, 25 or fewer, 20 or fewer, 15 or fewer, 12 or fewer, 10 or fewer, 8 or fewer, 7 or fewer, 6 or fewer, 5 or fewer, 4 or fewer, 3 or fewer, 2 or fewer, 1 sample, or no samples loaded thereon. Optionally, the thermal cycler may receive 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 25 or more, 30 or more, 35 or more, 40 or more, 50 or more, 100 or more, 150 or more, 200 or more, 250 or more, 300 or more, 350 or more, 400 or more, or 500 or more samples. The samples may be provided within reaction vessels that may be accepted by the thermal cycler. Alternatively, the sample may be directly provided to the thermal cycler without the reaction vessels, or may be loaded on reaction vessels built into the thermal cycler. The samples may be provided as one or more rows, one or more columns, an array, staggered rows or columns, concentric circles, randomly disposed, or any other configuration.

The thermal cycler 110 may have one or more wells. The wells may be configured to accept a reaction vessel or sample directly. The wells may be indentations on a support structure. In some instances, the support structure may be a heating/cooling block. For example, the wells may be formed directly into the heating unit itself. The reaction vessels may be inserted into the wells and may directly contact the heating unit. The reaction vessels and samples therein may experience conductive heating and cooling.

A reaction vessel can be part of an array of reaction vessels. An array of reaction vessels may be particularly useful for automating methods and/or simultaneously processing multiple samples 130a-d. For example, a reaction vessel may be a well of a microwell plate comprised of a number of wells. In another example, a reaction vessel may be held in a well of a thermal block of a thermal cycler, wherein the block of the thermal cycler comprises multiple wells each capable of receiving a sample vessel. An array comprised of reaction vessels may comprise any appropriate number of reaction vessels. For example, an array may comprise at least about 2, 4, 6, 8, 10, 12, 14, 16, 18, 20, 25, 35, 48, 96, 144, 384, or more reaction vessels. A reaction vessel part of an array of reaction vessels may also be individually addressable by a fluid handling device, such that the fluid handling device can correctly identify a reaction vessel and dispense appropriate fluid materials into the reaction vessel. Fluid handling devices may be useful in automating the addition of fluid materials to reaction vessels.

The reaction vessels may be individually movable relative to one another. The reaction vessels may be individually removable from the thermal cycler 110. Alternatively, reaction vessels may be connected to one another. In some instances, groups or strips of reaction vessels may be provided that may be collectively moved relative to other groups or strips of reaction vessels. Alternatively, the reaction vessels may be stationary relative to one another or relative to the rest of the thermal cycler.

As discussed, multiple samples 130a-d may be provided to the thermal cycler. The thermal cycler may simultaneously heat and cool the samples within the thermal cycler. Each of the samples may be controlled along the same temperature profile. Alternatively, different profiles may be provided for different samples. In some instances, the temperature profiles of the samples may be individually controllable, or controllable on a group by group or zone by zone basis. The thermal cycler may include a heating/cooling block that may have the same temperature throughout. Alternatively, a temperature gradient may be provided on the heating/cooling block. Different samples may be placed at different positions along the temperature gradient to yield different thermal cycling temperature profiles.

Each sample 130a-d may provide a signal that may be detectable by one or more detectors 120. Any description herein of a detector may apply to a single detector or multiple detectors. For example, if eight samples are provided, a single detector may detect signals from all eight samples, each sample may have its own detector (yielding a total of eight detectors), or multiple samples may be detected by a single detector, wherein multiple detectors may be provided overall. The detector may be capable of receiving optical signals from the samples during nucleic acid amplification of the samples. The detector may receive the optical signals simultaneously. The detector may receive optical signals from the samples continuously or on a periodic basis. In some instances, the detector may sequentially receive signals from the samples on a sequential or step-through basis.

A plurality of optical pathways 140a-d may be provided. In some instances, individualized optical pathways may be provided between the samples 130a-d and the detector 120. The optical pathways may preferably not interfere with one another. In some instances, the optical pathways may be optically isolated from one another. As previously described, optical pathways may include a line-of-sight between the samples and the detector. In one example, a single imaging device, such as a camera, may image the samples simultaneously. In other examples, optical pathways may include optical elements. For example, separate fiber optic pathways may be provided between each sample and the detector. The multiplexing of the samples and optical detectors may permit the device to load and amplify nucleic acid from multiple samples simultaneously.

Alternatively, a single optical pathway may be provided between a plurality of samples 130a-d and the detector 120.

As previously described, the thermal cycler may not have a detector built into the thermal cycler. The detector may be separate or separable from the thermal cycler. Real time detection of the signals from the sample may or may not occur for the thermal cycler.

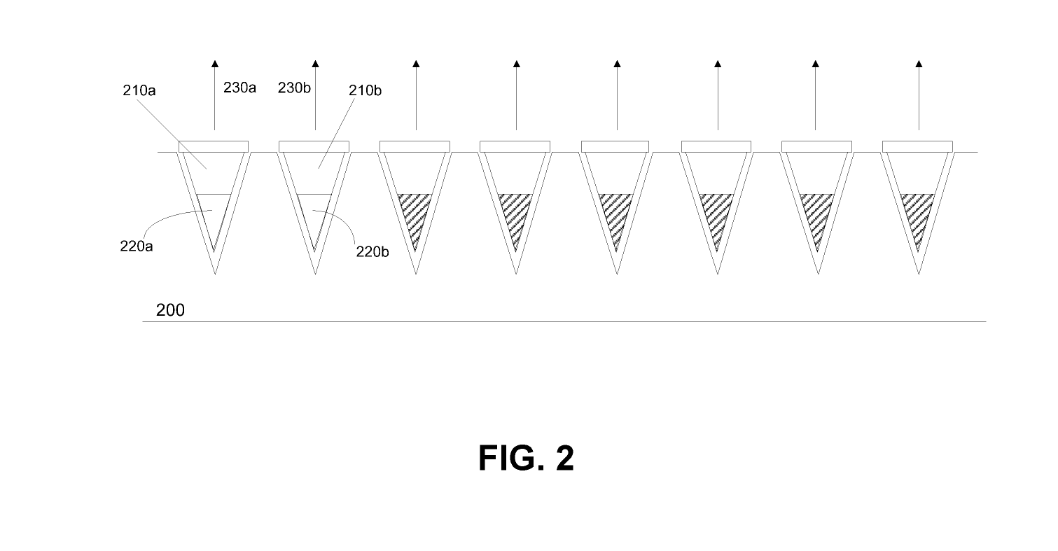

FIG. 2 shows samples that may be provided within sample containers supported by a device in accordance with an embodiment of the invention. The sample containers may be reaction vessels 210a, 210b. The reaction vessels may be supported by a supporting device 200. The supporting device may have one or more indentations into which the reaction vessels may be inserted. The indentations may be wells. The reaction vessels may contain samples 220a, 220b therein. Optical signals 230a, 230b may be emitted from the sample and may leave the reaction vessels.

The supporting device may have one or more indentations built therein. The support device may be a heating and cooling device. Any description of heating herein may also apply to both heating and cooling. In some instances, the support device may be heated using resistive conductive heating. In some instances, the support device may be Peltier device that may be capable of heating and cooling the sample therein. The support device may be a solid block or may include cavities, passageways, indentations, or other features. The support device may be formed from a metallic material. In some instances, the support device may be formed from a material of high thermal conductivity. The support device itself may be a heater, or may be in thermal communication with a heater. For example, the support device may be a thermally conductive block positioned on top of a heating block.

The support device may have any number of indentations therein. For example, the support device may include greater than or equal to about 1, 2, 3, 4, 5, 6, 7, 8, 10, 12, 14, 16, 18, 20, 24, 30, 35, 40, 48, 50, 60, 70, 80, 90, 96, 100, 120, 144, 150, 200, 250, 300, 384, 400, 500, 700, 1000, 1536, 2000 indentations. The support device may include fewer than or equal to about any of the number indentations described herein. In some instances, the number of indentations may fall in a range between any two of the values of described herein. The indentations may be sized and/or shaped to accept one or more reaction vessels 210a, 210b. The outer surface of the reaction vessels may contact interior surfaces of the indentation. The contact may be substantially flush so that the majority of the outer surface area of the reaction vessel contacts the indentation. This may improve thermal contact between the sample contained therein and a heating and cooling element.

The reaction vessels 210a, 210b may have any characteristic or dimension as described elsewhere herein. In some instances, all reaction vessels loaded into the thermal cycler may have the same characteristics. Otherwise, different types of reaction vessels may be loaded thereon. The support may be capable of accepting a single type of reaction vessel or multiple types of reaction vessels. For example, the support may be capable of accepting a flat topped reaction vessel and a dome topped reaction vessel. The support may be capable of accepting reaction vessels configured to contain different volumes of sample (e.g., 0.1 mL, 0.2 mL, 0.5 mL reaction vessels). The support may be capable of accepting reaction vessels of different heights (e.g., reaction vessels varying by more than about 0.1 mm, 0.5 mm, 1 mm, 1.5 mm, 2 mm, 2.5 mm, 3 mm, 4 mm, 5 mm, 6 mm, 7 mm, 8 mm, 9 mm, 10 mm, 12 mm, 15 mm, 20 mm, 25 mm, 30 mm, 40 mm, or 50 mm or more in height). The support may be capable of accepting reaction vessels of different cross-sectional sizes or shapes. The indentations on the support may all be filled with reaction vessels. Alternatively, one or more empty indentation may remain. A user may have an option of loading the reaction vessels thereon at the user's discretion.

The reaction vessels 210a, 210b may contain a sample 220a, 220b therein. The sample may have any characteristics as described elsewhere herein. The sample may be a reaction mixture that may include a target nucleic acid. The sample may also include a reporter agent and/or any other types of reagents needed for nucleic acid amplification. The samples within the reaction vessels may be from the same subject or from different subjects. The samples may be from the same type of subject (e.g., human or same type of animal) or from different types of subjects. The samples may be the same type of sample or may be different types of samples. For example, they may be different types of biological samples and/or collected from different portions of one or more subjects. The same amount of sample may be provided or varying amounts of sample may be provided.

Optical signals 230a, 230b may be provided from the sample 220a, 220b. The optical signals may leave the reaction vessels 210a, 210b. In some instances, the optical signals may leave via a top of the reaction vessel. In other instances, the optical signals may leave via a bottom or side of the reaction vessel. In some instances, optical elements may be built into the support that may aid in permitting optical signals to escape.

Any voltage value may be used for thermal cycling and/or detection. In some embodiments, low voltage may be used to for thermal cycling. In some embodiment, the low voltage may be less than or equal to about 60 V, 50 V, 48 V, 40 V, 30 V, 24 V, 20 V, 18 V, 16 V, 15 V, 14 V, 13 V, 12V, 11 V, 10V, 9 V, 8V, 7 V, 6 V, 5 V, 4 V, 3 V, 2 V, or 1 V to perform the thermal cycling. In some instances, the a low voltage of less than or equal to about 50 V, 40 V, 30 V, 24 V, 20 V, 18 V, 16 V, 15 V, 14 V, 13 V, 12V, 11 V, 10V, 9 V, 8V, 7 V, 6 V, 5 V, 4 V, 3 V, 2 V, or 1 V may be used to perform the combination of thermal cycling and detecting.

Any degree of power may be used for thermal cycling and/or detecting. In some instances, a low degree of power may be used for thermal cycling, or the combination of thermal cycling and detecting. For instance, about 84 W may be used to perform the thermal cycling and detecting. In some instances, a low power may be less than or equal to about 250 W, 200 W, 150 W, 130 W, 120 W, 110 W, 100 W, 90 W, 85 W, 84 W, 83 W, 80 W, 75 W, 70 W, 65 W, 60 W, 55 W, 50 W, 45 W, 40 W, 35 W, 30 W, 25 W, 20 W, 15 W, 10 W, 5 W, 1 W, 500 mW, 100 mW, 50 mW, 10 mW, 5 mW, or 1 mW. The amount of power used to operate the device may be less than or equal to any of the values described herein. Alternatively, the amount of power used to operate the device may be greater than equal to any of the values described herein. The amount of power used to operate the device may fall into a range between any two of the values described herein. The amount of power used to operate the thermal cycler and detector may have a total less than any of the values described herein. The amount of power used to operate the thermal cycler and detector may have a total greater than any of the values described herein. The amount of power used to operate the thermal cycler and detector may fall into a range between any two of the values described herein. The amount of power used to operate the thermal cycler may or may not include the amount of power to operate a lid of the thermal cycler, such as a heated lid of the thermal cycler.

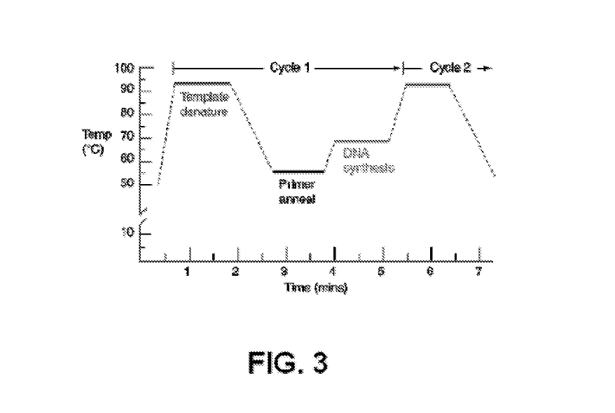

FIG. 3 shows an example of a thermal cycle in accordance with an embodiment of the invention. The thermal cycles may include heating and cooling of a sample. For example, there may be temperature for template denaturing, a temperature for primer annealing, and a temperature for DNA synthesis. The thermal cycler of the device may control the temperature to heat and cool to these temperatures. Temperature measurements are provided by way of example only and are not limiting. Similarly, amounts of time are provided by way of example only and are not limiting.

The thermal cycler may cause the sample to undergo any number of thermal cycles. The nucleic acid amplification may occur over the course of the multiple cycles. Examples of thermal cycling processes are provided as follows, and are not limiting. Any type of thermal cycling technique known in the art may be employed by the device.

In any of the various aspects, primer sets directed to a target nucleic acid may be utilized to conduct nucleic acid amplification reaction. Primer sets generally comprise one or more primers. For example, a primer set may comprise about 1, 2, 3, 4, 5, 6, 7, 8, 9, 10, or more primers. In some cases, a primer set or may comprise primers directed to different amplified products or different nucleic acid amplification reactions. For example, a primer set may comprise a first primer necessary to generate a first strand of nucleic acid product that is complementary to at least a portion of the target nucleic acid and a second primer complementary to the nucleic acid strand product necessary to generate a second strand of nucleic acid product that is complementary to at least a portion of the first strand of nucleic acid product.

For example, a primer set may be directed to a target RNA. The primer set may comprise a first primer that can be used to generate a first strand of nucleic acid product that is complementary to at least a portion the target RNA. In the case of a reverse transcription reaction, the first strand of nucleic acid product may be DNA. The primer set may also comprise a second primer that can be used to generate a second strand of nucleic acid product that is complementary to at least a portion of the first strand of nucleic acid product. In the case of a reverse transcription reaction conducted in parallel with DNA amplification, the second strand of nucleic acid product may be a strand of nucleic acid (e.g., DNA) product that is complementary to a strand of DNA generated from an RNA template.

Where desired, any suitable number of primer sets may be used. For example, about 1, 2, 3, 4, 5, 6, 7, 8, 9, 10, or more primer sets may be used. Where multiple primer sets are used, one or more primer sets may each correspond to a particular nucleic acid amplification reaction or amplified product.

In some embodiments, a DNA polymerase is used. Any suitable DNA polymerase may be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases.

A reverse transcriptase is used may be used in accordance with some embodiments of the invention. Any suitable reverse transcriptase may be used. A reverse transcriptase generally refers to an enzyme that is capable of incorporating nucleotides to a strand of DNA, when bound to an RNA template. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration.

Denaturation temperatures may vary depending upon, for example, the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations may vary depending upon, for example, the particular biological sample analyzed, the particular source of target nucleic acid (e.g., viral particle, bacteria) in the biological sample, the reagents used, and/or the desired reaction conditions. For example, a denaturation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures may vary depending upon, for example, the particular biological sample analyzed, the particular source of target nucleic acid (e.g., viral particle, bacteria) in the biological sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon, for example, the particular biological sample analyzed, the particular source of target nucleic acid (e.g., viral particle, bacteria) in the biological sample, the reagents used, and/or the desired reaction conditions. For example, an elongation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) necessary to obtain a detectable amplified product (e.g., a detectable amount of amplified DNA product that is indicative of the presence of a target RNA in a biological sample). For example, the number of cycles necessary to obtain a detectable amplified product (e.g., a detectable amount of DNA product that is indicative of the presence of a target RNA in a biological sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target RNA in a biological sample) may be obtained at a cycle threshold value (Ct) of less than 100, 75, 70, 65, 60, 55, 50, 45, 40, 35, 30, 25, 20, 15, 10, or 5.

The time for which amplification yields a detectable amount of amplified product indicative of the presence of a target nucleic acid amplified can vary depending upon the biological sample from which the target nucleic acid was obtained, the particular nucleic acid amplification reactions to be conducted, and the particular number of cycles of amplification reaction desired. For example, amplification of a target nucleic acid may yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a target RNA may yield a detectable amount of amplified DNA product indicative to the presence of the target RNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture may be subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, or all four of denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions could be used.

In some embodiments, the ramping time (i.e., the time the thermal cycler takes to transition from one temperature to another) and/or ramping rate can be important factors in amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification.

Optionally, the ramping time and/or ramping rate can be different between cycles. In some situations, however, the ramping time and/or ramping rate between cycles can be the same. The ramping time and/or ramping rate can be adjusted based on the sample(s) that are being processed.

In some situations, the ramping time between different temperatures can be determined, for example, based on the nature of the sample and the reaction conditions. The exact temperature and incubation time can also be determined based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

In some embodiments, a target nucleic acid may be subjected to a denaturing condition prior to initiation of a primer extension reaction. In the case of a plurality of series of primer extension reactions, the target nucleic acid may be subjected to a denaturing condition prior to executing the plurality of series or may be subjected to a denaturing condition between series of the plurality. For example, the target nucleic acid may be subjected to a denaturing condition between a first series and a second series of a plurality of series. Non-limiting examples of such denaturing conditions include a denaturing temperature profile (e.g., one or more denaturing temperatures) and a denaturing agent.

An advantage of conducting a plurality of series of primer extension reaction may be that, when compared to a single series of primer extension reactions under comparable denaturing and elongation conditions, the plurality of series approach yields a detectable amount of amplified product that is indicative of the presence of a target nucleic acid in a biological sample with a lower cycle threshold value. Use of a plurality of series of primer extension reactions may reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

In some embodiments, a biological sample may be preheated prior to conducting a primer extension reaction. The temperature (e.g., a preheating temperature) at which and duration (e.g., a preheating duration) for which a biological sample is preheated may vary depending upon, for example, the particular biological sample being analyzed. In some examples, a biological sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample may be preheated at a temperature from about 80° C. to about 110° C. In some examples, a biological sample may be preheated at a temperature from about 90° C. to about 100° C. In some examples, a biological sample may be preheated at a temperature from about 90° C. to about 97° C. In some examples, a biological sample may be preheated at a temperature from about 92° C. to about 95° C. In still other examples, a biological sample may be preheated at a temperature of about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

In any of the various aspects, the time required to complete the elements of a method may vary depending upon the particular steps of the method. For example, an amount of time for completing the elements of a method may be from about 5 minutes to about 120 minutes. In other examples, an amount of time for completing the elements of a method may be from about 5 minutes to about 60 minutes. In other examples, an amount of time for completing the elements of a method may be from about 5 minutes to about 30 minutes. In other examples, an amount of time for completing the elements of a method may be less than or equal to 120 minutes, less than or equal to 90 minutes, less than or equal to 75 minutes, less than or equal to 60 minutes, less than or equal to 45 minutes, less than or equal to 40 minutes, less than or equal to 35 minutes, less than or equal to 30 minutes, less than or equal to 25 minutes, less than or equal to 20 minutes, less than or equal to 15 minutes, less than or equal to 10 minutes, or less than or equal to 5 minutes.

The automated thermal cycler may be capable of controlling a temperature of a sample precisely to achieve a desired temperature profile. The automated thermal cycler may be capable of controlling the temperature to within about plus or minus 5 degrees C., 4 Degrees C., 3 degrees C., 2 degrees C., 1.2 degrees C., 1 degree C., 0.7 degrees C., 0.5 degrees C., 0.3 degrees C., 0.1 degrees C., 0.05 degrees C., 0.01 degrees C., 0.005 degrees C., or 0.001 degrees C. The automated thermal cycler may advantageously be capable of providing high quality temperature control while operating at a low voltage and/or low power. The automated thermal cycler may be advantageously capable of delivering high quality temperature control while having small dimensions. In some instances, heat blocks may be used. Liquid metal heat blocks may be an example of heat blocks that may be used. A heating system using thermally conductive fluid may optionally be used. Alternatively, no thermally conductive fluid may be used. In some instances, a high density of heating and/or cooling elements may be provided for a heat block.

Detection of signals from the sample undergoing amplification may occur throughout the process. The detection may occur continuously or at one or more points during the amplification process. The sample may emit optical signals throughout the process. The optical signals may be related to the amount of amplified target nucleic acid in the sample. In other implementations, detection of signals from the sample may occur after amplification has been completed. Optionally, detection of signals from the sample may not occur while the amplification and/or thermal cycling is in process.

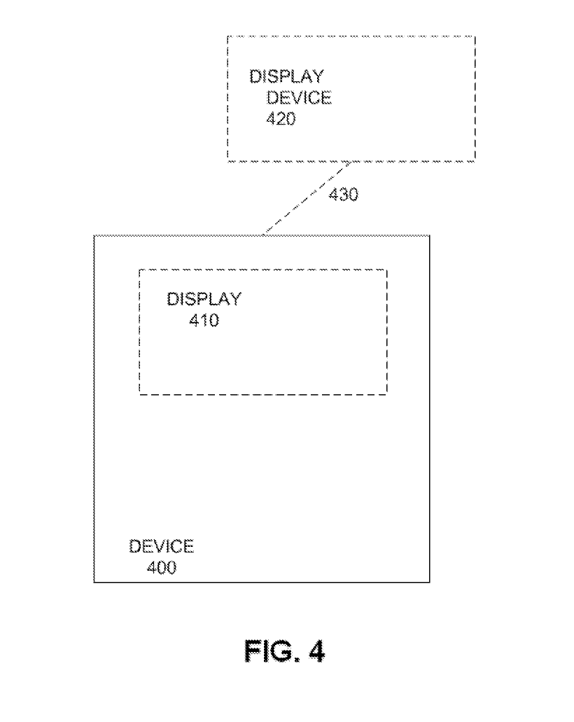

FIG. 4 shows an example of a device and a display in accordance with an embodiment of the invention. The device 400 may be used to conduct nucleic acid amplification of a target nucleic acid in a sample. The sample may be loaded into the device. A thermal cycler of the device may alternating heat and cool the sample. The device may include a detector that may be capable of detecting signals relating to the amplification of the target nucleic acid in the sample in real-time.

Optionally, data relating to the detected signals may be displayed in real-time. For example, data relating to the progress of the nucleic acid amplification and/or results of the nucleic acid amplification may be displayed while amplification is occurring. In some instances, a display 410 may be built-into the device. For example, the display may be provided on a housing of the device. Any description of a display may apply to any type of output module. The display may include a visual display, as well as optional audio or tactile output of information. The display may show information on a screen or other type of user interface (UI). For example, a screen may be built into the device.

In other instances, the data may be shown on a separate display device 420. The separate display device may communicate with the device 400. In some instances, communications may occur via a connection 430. The connection may be a hard-wired connection or a wireless connection. Direct communications may occur between the device and the display device. For example, Bluetooth, infra-red communications, radio, WiFi, or other direct communications may occur. In other instances, indirect communications may occur between the device and the display device. For examples, communications may occur over a network, such as a local area network (LAN), or wide area network (WAN) such as the Internet. In some instances, telecommunications networks may be used (e.g., cellular phone networks, data networks). In some examples, 3G or 4G networks may be used for communications. One or more intermediate devices, such as relay devices (e.g., towers) or router, may be used in communications. Alternatively, no intermediate devices may be used.

A device 400 may have an input module that receives a user request to amplify a target nucleic acid (e.g., target RNA, target DNA) present in a biological sample obtained direct from a subject. Any suitable module capable of accepting such a user request may be used. The input module may comprise, for example, a device that comprises one or more processors. The input module may be built into the device. The input module may be integrated into a housing of the device or accessible from outside the housing.

Alternatively, the input module may be separate or separable from the device. The input module may communicate with the device over a connection, such as those described elsewhere herein. Non-limiting examples of devices that comprise processors include a desktop computer, a laptop computer, a tablet computer (e.g., Apple® iPad, Samsung® Galaxy Tab), a cell phone, a smart phone (e.g., Apple® iPhone, Android® enabled phone), a personal digital assistant (PDA), a video-game console, a television, a music playback device (e.g., Apple® iPod), a video playback device, a pager, and a calculator. Processors may be associated with one or more controllers, calculation units, and/or other units of a computer system, or implanted in firmware as desired. If implemented in software, the routines (or programs) may be stored in any computer readable memory such as in RAM, ROM, flash memory, a magnetic disk, a laser disk, or other storage medium. Likewise, this software may be delivered to a device via any known delivery method including, for example, over a communication channel such as a telephone line, the internet, a local intranet, a wireless connection, etc., or via a transportable medium, such as a computer readable disk, flash drive, etc. The various steps may be implemented as various blocks, operations, tools, modules or techniques which, in turn, may be implemented in hardware, firmware, software, or any combination thereof. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

In some embodiments, the input module is configured to receive a user request to perform amplification of the target nucleic acid. The input module may receive the user request directly (e.g. by way of an input device such as a keyboard, mouse, or touch screen operated by the user) or indirectly (e.g. through a wired or wireless connection, including over the internet). Via output electronics, the input module may provide the user's request to the amplification module. In some embodiments, an input module may include a user interface (UI), such as a graphical user interface (GUI), that is configured to enable a user provide a request to amplify the target nucleic acid. A GUI can include textual, graphical and/or audio components. A GUI can be provided on an electronic display, including the display of a device comprising a computer processor. Such a display may include a resistive or capacitive touch screen.

Non-limiting examples of users include the subject from which the biological sample was obtained, medical personnel, clinicians (e.g., doctors, nurses, laboratory technicians), laboratory personnel (e.g., hospital laboratory technicians, research scientists, pharmaceutical scientists), a clinical monitor for a clinical trial, or others in the health care industry.

As previously described, the system comprises an output module operatively connected to the amplification module. In some embodiments the output module may comprise a device with a processor as described above for the input module. The output module may include input devices as described herein and/or may comprise input electronics for communication with the amplification module. In some embodiments, the output module may be an electronic display, such as a display 410 on a nucleic acid amplification device or a separate display device 420. In some cases, the electronic display may comprise a UI. In some embodiments, the output module is a communication interface operatively coupled to a computer network such as, for example, the internet. In some embodiments, the output module may transmit information to a recipient at a local or remote location using any suitable communication medium, including a computer network, a wireless network, a local intranet, or the internet. In some embodiments, the output module is capable of analyzing data received from the amplification module. The output module may analyze information in real-time while amplification is occurring. Some data may be analyzed after the amplification has been completed. In some cases, the output module includes a report generator capable of generating a report and transmitting the report to a recipient, wherein the report contains any information regarding the amount and/or presence of amplified product as described elsewhere herein. In some embodiments, the output module may transmit information automatically in response to information received from the amplification module, such as in the form of raw data or data analysis performed by software included in the amplification module. Alternatively, the output module may transmit information after receiving instructions from a user. Information transmitted by the output module may be viewed electronically or printed from a printer.

One or more of the input module, amplification module, and output module may be contained within the same device or may comprise one or more of the same components. For example, an amplification module may also comprise an input module, an output module, or both. In other examples, a device comprising a processor may be included in both the input module and the output module. A user may use the device to request that a target nucleic acid be amplified and may also be used as a means to transmit information regarding amplified product to a recipient. In some cases, a device comprising a processor may be included in all three modules, such that the device comprising a processor may also be used to control, provide instructions to, and receive information back from instrumentation (e.g., a thermal cycler, a detector, a fluid handling device) included in the amplification module or any other module.

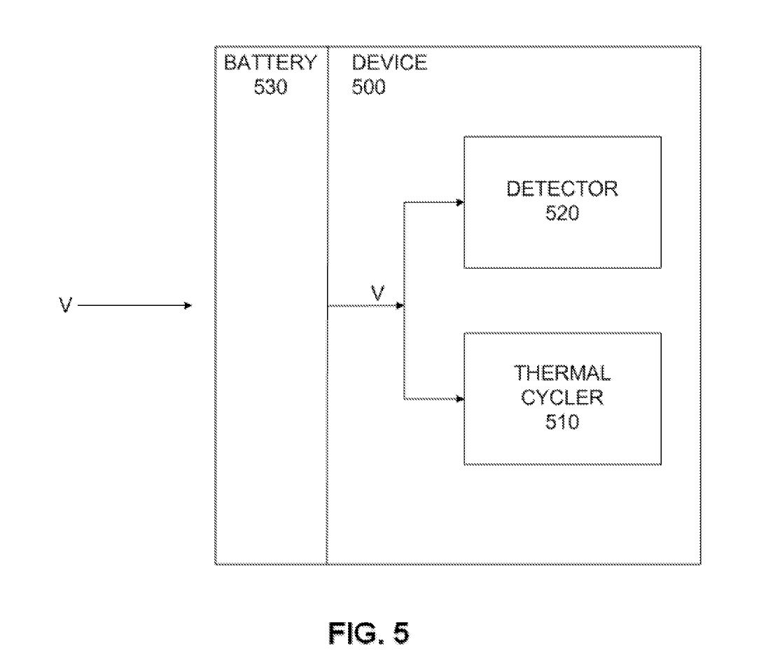

FIG. 5 shows an example of power being provided to a device that includes a battery in accordance with an embodiment of the invention. A device may include a thermal cycler 510 and detector 520 as described elsewhere herein. The device may also be operably linked to an energy storage device 530.

The energy storage device 530 may be a battery pack. The battery pack may be a portable battery pack. The battery pack may comprise one or more batteries. The batteries may be an electrochemical energy storage device. For example, the battery pack may include a single or multiple battery cells. The battery may be a lithium-based battery, such as a lithium ion battery. The battery may have any chemistry, including but not limited to lead acid batteries, valve regulated lead acid batteries (e.g., gel batteries, absorbed glass mat batteries), nickel—cadmium (NiCd) batteries, nickel-zinc (NiZn) batteries, nickel metal hydride (NiMH) batteries, or lithium-ion (Li-ion) batteries.

The energy storage device 530 may be part of the device 500. In one example, the energy storage device may be provided within a housing of the device. The energy storage device may be removable from the device or may be an integral part of the device. In some instances, the energy storage device may be placed within the housing of the device and/or removed from within the housing of the device. Energy storage devices may be swapped or exchanged. In some instances, the energy storage devices may be rechargeable. The energy storage devices may be rechargeable while within the device, or may be removed to be recharged.

In another example, the energy storage device may be directly attached to the device but not within the housing of the device. For example, an external attachment and/or connection may be provided. The energy storage device may directly contact the device housing. The energy storage device may be attached to the device and into place via one or more connector or mechanical fastener. The energy storage device may be separably attached to the device. For example, the energy storage may be attached and detached from the device. Energy storage devices may be swapped. The energy storage device may be rechargeable. The energy storage devices may be rechargeable while attached to the device, or may be separated to be recharged.

The energy storage device may be electrically connected to the device via one or more connector. For example, the connector may be a wire, cable, or other conductive pathway. Optionally, the connector may be a flexible conductive pathway. For example, the energy storage device may be plugged into the device or vice versa. The energy storage device and the device may be separable from one another. Different energy storage devices may be swapped for the device. For example, the device may plug into different energy storage devices. The energy storage device may be rechargeable. The energy storage devices may be rechargeable while electrically connected to the device, or may be separated to be recharged. A physical electrical connection may be provided between the energy storage device and the device. Alternatively, the energy storage device may wirelessly power the device.

The energy storage device may use any voltage V to power the device. In some examples, the energy storage device may use low voltage to power the device. For example, the energy storage device may provide no more than 48, 24, or 12 V or other voltage values described elsewhere herein to power the device. The storage device may use no more than a total of 48, 24, or 12 V (or any other voltage value described elsewhere herein) to power the thermal cycler and the detector of the device. Optionally, other components of the device (e.g., input module, output module, light source, processors), may also be powered using no more than a total of 48, 24, or 12 V.

The energy storage device may receive a low voltage power when charging the device. For example, no more than 48, 24, or 12 V, or other voltage values described elsewhere herein, may be used to charge the energy storage device. The energy storage device may optionally output energy at the same voltage as it receives.

In some instances, when energy is coming in from an external power source, the device may be powered directly from the external power source. In another example, even when energy is coming in from an external power source, the device may be powered through the energy storage device, and the external power source may be used to charge the energy storage device. In some instances, the energy coming in from the external power source may be used to power the device when the energy storage unit is fully charged.

As previously described any low voltage power may be used to power the device. Similarly, any low voltage power may be used to charge the energy storage device. Any reference to low voltage may include a voltage of 50 V or less, 40 V, or less, 35 V, or less, 30 V, or less, 25 V or less, 24 V or less, 22 V or less, 20 V or less, 19 V or less, 18 V or less, 17 V or less, 16 V or less, 15 V or less, 14 V or less, 13.5 V or less, 13 V or les, 12.5 V or less, 12 V or less, 11.5 V or less, 11 V or less, 10.5 V or less, 10 V or less, 9.5 V or less, 9 V or less, 8 V or less, 7 V or less, 6 V or less, 5 V or less, 4 V or less, 3 V or less, 2 V or less, 1 V or less, 500 mV or less, 200 mV or less, 100 mV or less, 50 mV or less, 10 mV or less, 5 mV or less, or 1 mV or less.

The device may be capable of operating at low power. Any combination of components may be capable of operating at low power. For example, the thermal cycler and the detector may be capable of operating at a combined low power. The thermal cycler and detector and input unit may be capable of operating at a combined low power. The thermal cycler, detector, input unit and output unit may be capable of operating at a combined low power. Any reference to a low power may include a power of 250 W or less, 200 W or less, 150 W or less, 130 W or less, 120 W or less, 110 W or less, 100 W or less, 90 W or less, 85 W or less, 84 W or less, 83 W or less, 80 W or less, 75 W or less, 70 W or less, 65 W or less, 60 W or less, 55 W or less, 50 W or less, 45 W or less, 40 W or less, 35 W or less, 30 W or less, 25 W or less, 20 W or less, 15 W or less, 10 W or less, 5 W or less, 1 W or less, 500 mW or less, 100 mW or less, 50 mW or less, 10 mW or less, 5 mW or less, 1 mW or less, or any other power value described elsewhere herein.

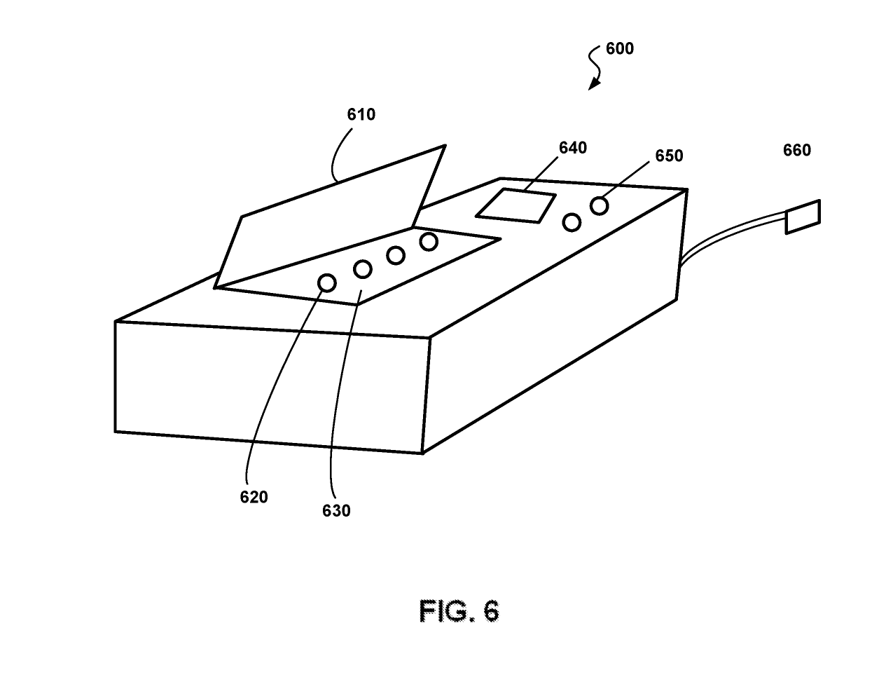

FIG. 6 shows an example of a device for conducting a nucleic acid amplification reaction in accordance with an embodiment of the invention. The device 600 may have a housing that may enclose one or more components of the device. A battery pack may be enclosed within the housing, on an exterior surface of the housing, or may be separate from the rest of the device.

The device may optionally have a lid 610. The lid may open to provide access to a support 630 which may be capable of receiving one or more samples 620. In some instances, the lid may be capable of moving between an open position and a closed position. Optionally, the lid may traverse one or more intermediary positions between the open and the closed position. During the closed position, the samples may be entirely enclosed within the housing. A user may not be able to access the samples when the lid is closed. The user may optionally not be able to see the samples and/or sample containers when the lid is closed. Alternatively, the user may see the samples and/or sample containers through the lid when the lid is closed. The samples may be at least partially isolated from the ambient environment when the lid is closed. The lid may lie flat over the samples while in the closed position. The lid may optionally form a portion of the housing. The samples may not be removed or added to the device when the lid is in the closed position. During the open position, the samples or samples containers may be exposed to the ambient environment. Samples may be removed or added to the device when the lid is in the opened position. A user may be able to access the samples when the lid is opened. A user may be able to see the samples and/or sample containers when the lid is opened.

The lid may open and/or close in response to an input from a user. The input from a user may include a manual manipulation of the device. The manual manipulation of the device may include contacting the lid directly, or contacting another portion of the device that may allow the lid to open and/or close. The other portion of the device may include a button, switch, slider, joystick, lever, touchpad, touchscreen, knob, or any other mechanism that may accept a user input. The input from the user may be provided via a device controller than may be remote to the device.

The support 630 may be used to heat and/or cool the samples. The support may alternatingly heat and cool the samples in accordance with a temperature profile having one or more thermal cycles. The same support may be used to heat and cool the samples, alternatively multiple supports may be used to heat and cool the samples. One or more supports may individually or collectively heat and cool the samples. The temperature may be any temperature profile, including those described elsewhere herein.

The temperature control may be provided in accordance with pre-programmed instructions. In some instances, the temperature control may be provided in accordance with non-transitory computer readable media comprising code, logic, or instructions to perform the steps for the temperature control. In one aspect, a computer readable medium may comprise machine executable code that, upon execution by one or more computer processors, implements a method of amplifying a target ribonucleic acid (RNA) present in a biological sample obtained from a subject, the method comprising: (a) providing a reaction vessel comprising the biological sample and reagents necessary for conducting nucleic acid amplification, the reagents comprising (i) a DNA polymerase and optionally a reverse transcriptase, and (ii) a primer set for the target nucleic acid, to obtain a reaction mixture; and (b) subjecting the reaction mixture in the reaction vessel to a plurality of series of primer extension reactions to generate amplified product from the target nucleic acid, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

Computer readable medium may take many forms, including but not limited to, a tangible (or non-transitory) storage medium, a carrier wave medium, or physical transmission medium. Non-volatile storage media include, for example, optical or magnetic disks, such as any of the storage devices in any computer(s) or the like, such as may be used to implement the calculation steps, processing steps, etc. Volatile storage media include dynamic memory, such as main memory of a computer. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution

Optionally a detector may be provided for the device. The detector may optionally be provided within a housing of the device. The detector may be capable of detecting optical signals from the samples. The detector may be capable of detecting optical signals while the lid is closed. The detector may be capable of detecting optical signals while the samples or sample containers are not exposed to an exterior of the device. The detectors may be capable of detecting optical signals while it is not possible to remove or add samples to the device. The detector may be capable of detecting optical signals while the support is heating and cooling the samples. The detector may be capable of detecting optical signals while nucleic acid amplification is occurring within the sample. Detection may occur in accordance with non-transitory computer readable media.

The device may include a display 640 in some embodiments of the invention. The display may include information about operation of the device and/or status of the operation of the device. The display may or may not include information about the progress of the nucleic acid amplification. In some instances, the display may include some information generated based on information received from a detector. This may include real-time information from the detector during the nucleic acid amplification.

One or more controls 650 may be provided. The one or more controls may permit a user to control the device. The controls may be separate from a display or may be integrated into a display. For example, the display may include a touchscreen that may be capable of both displaying information and accepting user input. The controls may accept tactile input, verbal input, and/or visual input (e.g., motions or gestures). The controls may accept a user input to turn the device on or off. The controls may accept user input to initiate a thermal cycling mode or select a thermal cycling mode from a plurality of options. The user may specify details relating to the thermal cycling modes. The user may provide input about detection of the nucleic acid amplification. The user may provide input about display and or transmittal of data resulting from detection of nucleic acid amplification. The user may or may not put information about different energy modes and/or energy storage modes. Display and/or control of the device may occur in accordance with non-transitory computer readable media.

The device may include a power connector 660. The power connector may be used to connect the device to a power source. The power source may be an on-grid power source or off-grid power source. The power source may be a vehicle, such as a passenger vehicle. The power source may be an energy storage device, such as a battery pack described elsewhere herein. The power connector may include a plug, pin, prongs, or other form of electrical connector. The power connector may be capable of receiving a low voltage amount to power the device.

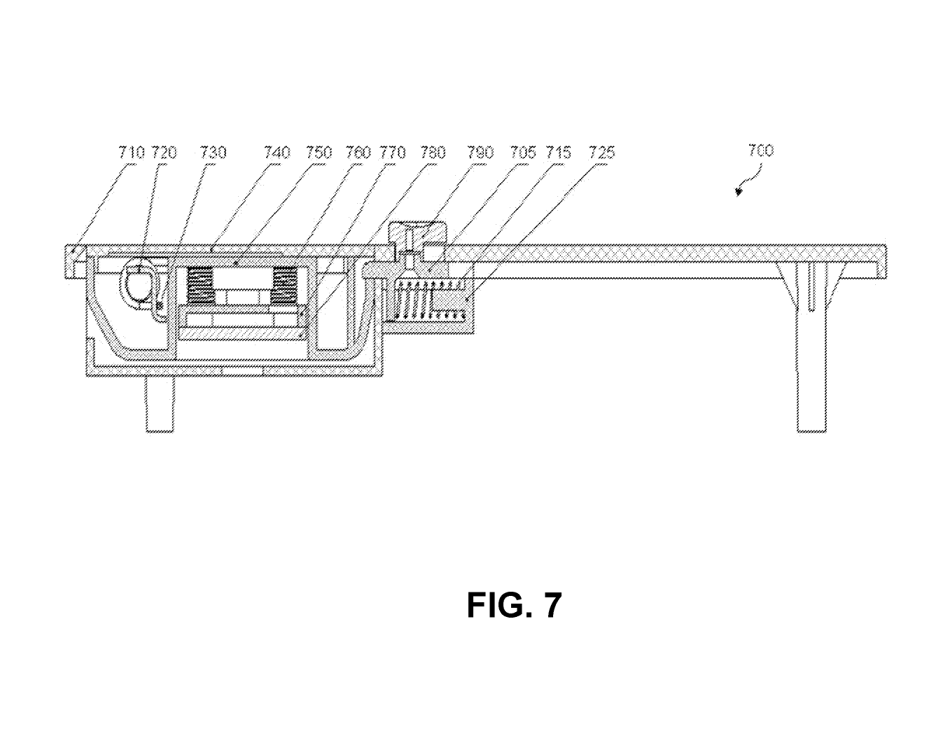

FIG. 7 shows an example of a lid configuration for a device in accordance with an embodiment of the invention. The lid configuration as illustrated may be provided for a thermal cycler, such as a thermal cycler having one or more of the previously described characteristics. The lid configuration may be integrated onto a surface or housing of the device.

The lid assembly 700 may include a heater 780. Any description of a heater may apply to any type of temperature control device, which may be used for heating and/or cooling. For instance, the heater of the lid may be capable of both heating and cooling. The heater may have a plate configuration. For example, the heater may be a heating plate. The heater may be embedded as part of the lid assembly.

The lid assembly may include an instrument panel 710. The instrument panel may be part of the housing or outer surface of the thermal cycler. The instrument panel may optionally accept an input from a user. The input from a user may be used to control the lid assembly 700, the heater 780, a supporting element that may support a sample container, an internal heater, a detector, and/or any other component of the thermal cycler.

A heater panel 740 may be a portion of the lid assembly that may form an outer surface of the lid assembly. The outer surface of the heater panel may be in line with an outer surface of the instrument panel 710. The heater panel and/or instrument panel may be in line with an outer surface of the device. The heater panel may or may not be in line with a contour of the device outer surface when the lid is closed. The heater panel may lie flat so that the lid does not substantially protrude from the device when the lid is closed. The heater panel may be a portion of the lid assembly that moves between an open position and a closed position. The heater panel may pivot about an axis to move between an open position and a closed position.

A torsion spring 730 may be provided to affect the movement of the heater panel 740 relative to the instrument panel 710. The heater panel may rotate about an axis of rotation relative to the instrument panel. The axis of rotation may be provided through a heater shaft. The heater shaft may be connected through its axial direction and further connected with the instrument panel. The torsion spring may be provided around the axis of rotation. In some examples, the torsion springs may be installed onto two symmetrical heater shafts. The torsion spring may effect a force on the heater panel. In some instances, the torsion spring provide a force on the heater panel for the heater panel to remain in an open position. A user may counteract the force provided by the torsion spring by pressing down on the heater panel, to cause it to lie flat against the instrument panel. Optionally a latching mechanism may be provided that may cause the heater panel to remain closed. When the latching mechanism is released, the torsion spring may cause the heater panel to move toward an open position. Optionally, a limit frame 720 for the torsion spring may be provided. The limit frame may cause the heater panel to stop at a particular position when being pushed by the torsion spring to an open position. The limit frame may limit the degree to which the heater panel may be opened. Any description herein of a torsion spring may apply to any other type of force inducing mechanism. The force inducing mechanism may be configured to provide a force on the heater panel to remain in an open position. The force inducing mechanism may provide a force that may be exerted about an axis of rotation. The force inducing mechanism may be an axial force inducing mechanism that may bias the heater panel in a particular direction about an axis of rotation. This bias or force may be counteracted by manual manipulation by the user and/or a latch or other mechanism that may be counter to the direction about the axis of rotation.

A heater bottom panel 750 may be provided. The heater bottom panel may be supported relative to the heater panel 740 and may move with the heater panel. The heater bottom panel may optionally be stationary relative to the heater panel. The heater bottom panel may be substantially parallel to the heater panel. The heater bottom panel may be at an angle relative to the heater panel. Optionally, the angle does not change while the lid is move between an open and closed position.

Optionally, the heater 780 may fit beneath a portion of the heater bottom panel and/or between two vertical portions of the heater bottom panel. The vertical portions of the heater bottom panel may aid in guiding the heater. The heater may be supported by one or more compression springs 760 or other force inducing members. Any description herein of compression springs may be applied to any type of force inducing member. The force inducing member may be a linear force inducing member that provides force in a linear direction (e.g., as opposed to a rotational/axial direction). The force inducing members may supply a force to the heater away from the outer surface of the lid. The force inducing member may provide a force to the heater downwards when the lid is closed. The force inducing member may provide a force to the heater toward one or more samples when the lid is closed. The force exerted by the compression spring may be counteracted by an object pressing on the heater. For instance, the object may be a cap/lid of a sample container when the lid is closed. The compression springs may permit the heater to move relative to the heater panel. The heater may optionally remain substantially parallel to the heater panel when moving relative to the heater panel. The heater may optionally provide a translated motion when moving while providing little or no rotational motion. The compression springs may provide a range of vertical motion for the heater. The compression springs may be a force inducing component that may induce a force on the heater to be at a distance from the heater bottom panel 750 horizontal component that corresponds to a natural length of the compression spring. The springs may be compressed to cause the heater to be at a distance closer to the heater bottom panel.

Optionally, a heating plate stand 770 may be provided which may aid in supporting the heater 780. The heating plate stand may be provided between the heater and the one or more compression springs 760 or other type of force inducing component. In some embodiments, the heater and the heating plate stand may be fixed onto the bottom panel 750 by connecting screws.

A switch 790 may be provided on the device. The switch may overlie a portion of the instrument panel 710 and/or the heater panel 740 (exterior surface of the movable lid). The switch may be connected to a buckle 705. The buckle may underlie the switch and may be configured to move with the switch. The buckle may include a protrusion that may come into contact with a portion of a heater bottom panel 750. A switch spring 715 or other force inducing component and a spring compression plate 725 may be provided. Any description of a switch spring may apply to any type of force inducing component that may provide a force against the buckle. The switch spring may contact the buckle. The switch spring may provide a force against the buckle. This may cause the protrusion on the buckle to overlie the portion of the heater bottom panel. A user may move the switch to counteract the force provided by the switch spring. Moving the switch away from the heater panel may cause the switch spring to compress. Moving the switch away from the heater panel may counteract the force provided by the switch spring. Moving the switch away from the heater panel may also cause the buckle to move correspondingly. The movement of the buckle away from the heater panel may cause the protrusion of the buckle to no longer cover the portion of the heater bottom panel. This may permit the torsion spring 730 to cause the heater panel to open. Thus, a user may move the switch away from the heater panel to cause the heater panel to open. The switch may optionally be move laterally with respect to the heater panel surface. In some instances, the switch may be laterally moved in a direction away from the heater panel, to permit the heater panel to open.

Any description of the switch may apply to any other type of mechanism (e.g., button, latch, lever, joystick, slider, knob, touchpad, touchscreen) that may permit a user to release a lid. The switch may be a lid-releasing mechanism. Manipulation of the lid-releasing mechanism by the user may cause the lid to move from a closed position to an open position. The lid-releasing mechanism may permit the lid to automatically open with aid of a force inducing component. Interaction with the lid-releasing mechanism may remove a force that was keeping the lid closed, and may permit the lid to open. An open lid may be a natural state of the lid, that may be counteracted by a user pressing down on the lid to close the lid, or a lid-holding mechanism, which may be released when the lid-releasing mechanism is used. When a lid is open, it may be pressed closed by a user, which may permit the lid-holding mechanism to engage when the lid reaches its closed state. When the user removes the user-induced force, the lid may remain closed, held by the lid-holding mechanism, until the lid-releasing mechanism is used.

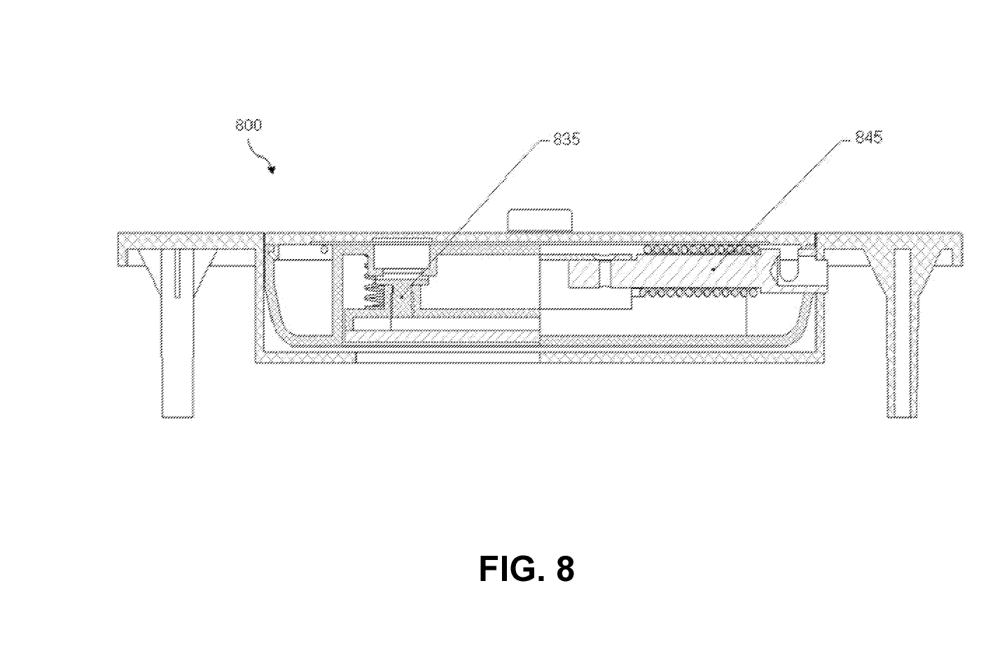

FIG. 8 shows an additional view of a lid configuration 800 for a device in accordance with an embodiment of the invention. As previously described, a screw 835 may be used to connect the heating plate and the heater stand to the heater bottom panel.

Furthermore, the heater shaft 845 may be supported by an instrument panel. One or more torsion springs may be supported around the heater shaft, which may affect the movement of the heater panel (lid). The heater shaft may or may not pass all the way through along the width of the heater panel. Alternatively, the heater shaft may be provided only part-way through. In some embodiments, multiple heater shafts may be provided. A heater shaft may provide a pivot around which the lid may rotate when the lid is moving between an open position and a closed position.

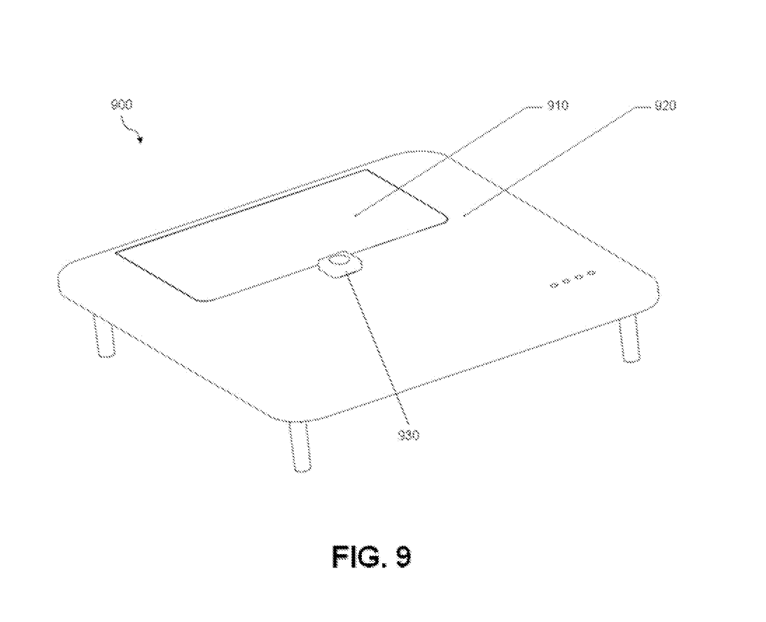

FIG. 9 shows an exterior view of a lid and instrument panel for a device in accordance with an embodiment of the invention. A lid assembly 900 may be provided. The lid assembly may include the lid 910 (which may also be referred to as a heater panel) and an instrument panel 920. The lid and/or instrument panel may be provided on a device, such as a thermal cycler. The lid and/or instrument panel may be provided on a housing of the device or integrated into the housing of the device. The lid and/or instrument may be exposed as part of the outer surface of the device. The lid assembly may have an embedded structure, and may be highly integrated. The lid assembly may optionally have a small footprint which may make it compatible with many portable devices that may require a heater. The lid assembly may be coupled to different types of devices that may utilizes a lid that may open and close, and which may include a heater embedded therein. For instance, the lid assembly may be provided on different types of thermal cycler devices, or other devices that need not be thermal cycler devices. The lid assembly may be provided as a single unit, or the lid and instrument panel may be provided as separate units.

In some instances, the footprint of the lid assembly may be less than or equal to about 1 cm², 5 cm², 10 cm², 15 cm², 20 cm², 25 cm², 30 cm², 40 cm², 50 cm², 60 cm², 70 cm², 80 mm², 90 cm², 100 cm², 110 cm², 120 cm², 130 cm², 140 cm², 150 cm², 160 cm², 170 cm², 180 cm², 190 mm², 200 cm², 220 cm², 250 cm², 300 cm², 400 cm², or 500 cm². The lid assembly may be installed on a portable device. The lid assembly may be installed on a device having any of the dimensions described elsewhere herein.

A switch 930 may optionally be provided. The switch may be used to cause the lid 910 to open. Moving the switch laterally may cause the lid to open. For example, sliding the switch away from the lid may cause the lid to open. As previously described, any description of a switch may apply to any type of mechanism that may cause a lid to open and/or close. For instance, the switch may be a lid-releasing mechanism that may cause the lid to open. The lid may automatically open when the switch is manipulated to release the lid. The lid may be forced closed, and a lid-holding portion may engage to keep the lid closed. The lid-holding portion may be operatively coupled to the lid-releasing mechanism so that manipulating the lid-releasing mechanism may cause the lid-holding mechanism to release its hold on the lid.

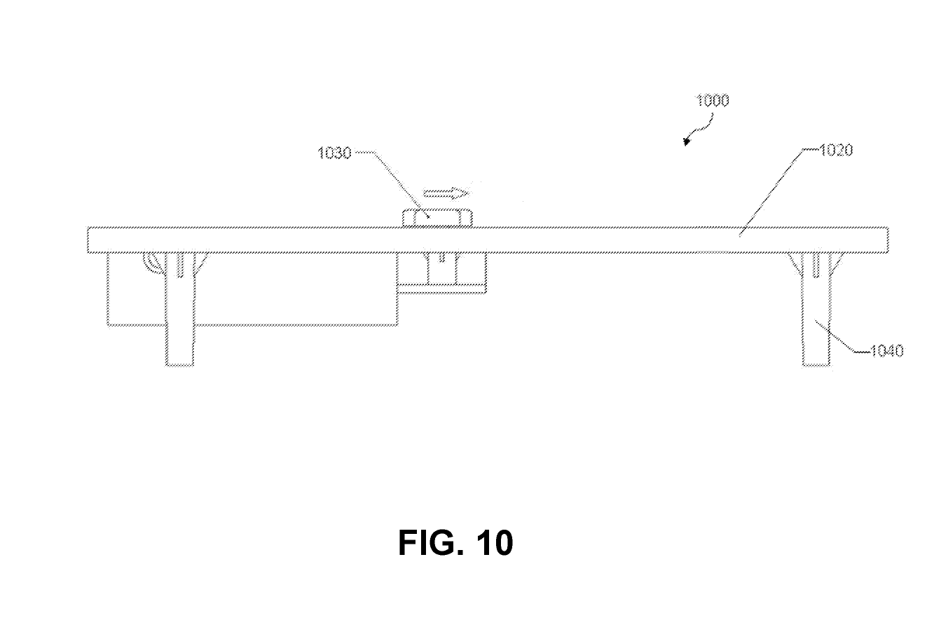

FIG. 10 provides an additional view of a lid configuration for a device in accordance with an embodiment of the invention. A side view may be provided for a lid assembly 1000. The lid assembly may have a lid and an instrument panel 1020. A movable switch 1030 may be provided. The switch may move along a limited range of motion. Optionally, one or more connectors 1040 may be provided for the lid assembly. The connectors may be used to attach the lid assembly to a device.

A lid of the lid assembly may be opened and/or closed. The lid of the lid assembly may move between an open position and a closed position. An opening/closing operation may utilize one or more of the following steps:

(1) Slide the switch 1030 backwards to release the lid. The switch may move a limited distance. In some instances, the switch may move less than or equal to about 1 mm, 2 mm, 3 mm, 4 mm, 5 mm, 6 mm, 7 mm, or 1 cm to release the lid. The switch may move more than any of the distances described herein. The switch may move in a range of distances between any two of the values provided herein. In alternate embodiments, any type of manipulation of a lid-releasing mechanism may be provided, which may include sliding of a component, flipping of a switch or lever, rotating or twisting a component, touching a surface, gestures, verbal commands, or any combination thereof.

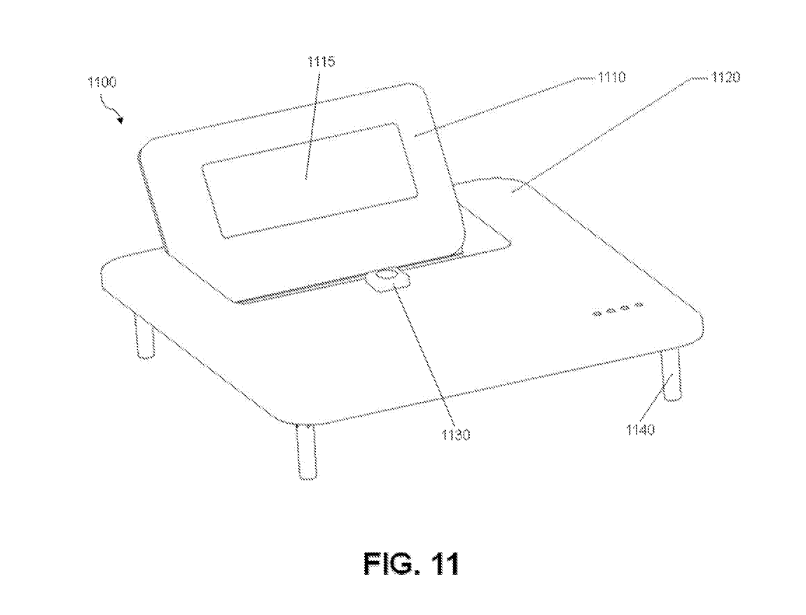

(2) A buckle coupled to the switch may move backwards as well, corresponding to the movement of the switch. The amount of backward movement by the buckle may be the same as, or may correspond to the amount of backward movement by the switch. When the buckle has moved a sufficient distance, a torsion spring may start to function. The buckle may optionally be a lid-holding mechanism. Moving the buckle may cause the buckle to no longer exert a force or control over the lid, which may enable to the torsion spring (e.g., axial force inducing component) to come into effect. It may cause the lid to spring open. The lid that springs open may include an outer surface (e.g., heater panel), heater, and/or compression springs. The lid may be opened to a predetermined angle. A stopper or other mechanism may be provided that may cause the lid to stop after it has opened to the predetermined angle. The heater may maintain its position at the predetermined angle without requiring an external force. For example, a torsion spring may exert a force on the heater to remain open, and a limit frame of the torsion spring may cause the heater to remain at the maximum open position. FIG. 11 shows a view of a lid 1110 in an open position in accordance with an embodiment of the invention. The lid may have a heater 1115. Optionally, the lid may move with the heater. When the lid rotates about an axis, the heater may move to rotate about the corresponding axis. The lid may be open relative to the instrument panel 1120 when the switch 1130 has been moved sufficiently backward. Optionally, one or more connector 1140 may be provided that may connect the lid assembly to a device.

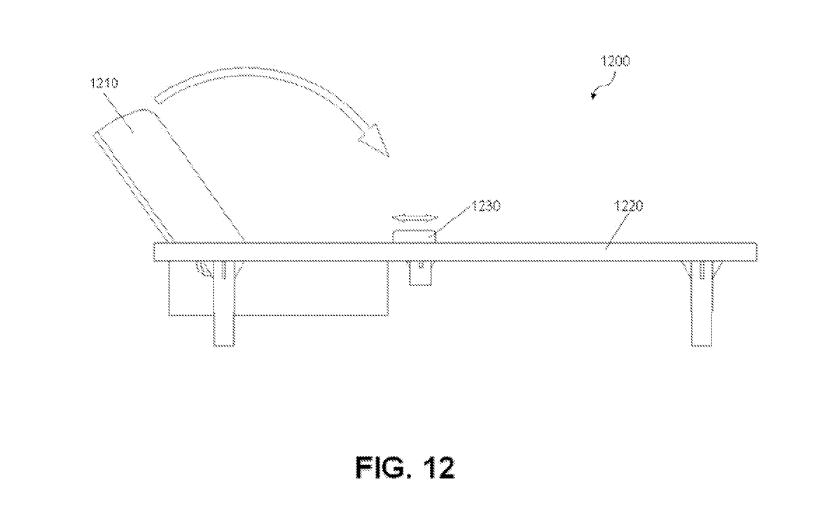

(3) A user may press the lid to close the heater. FIG. 12 shows an additional view of a lid 1210 in an open position in accordance with an embodiment of the invention. A user may press the lid downward toward the instrument panel 1220 to close the lid/heater. The user pressing the lid downwards may counteract the force provided by an axial force inducing component (e.g., torsion spring). A switch 1230 may be provided. The switch may move laterally relative to the instrument panel. When a lid is pressed down to be closed, the switch may slide into position to keep the lid closed. The switch may slide to keep the lid closed without requiring manipulation by the user. A force inducing component may be provided on the switch which may bias the switch to the position that would keep the lid closed. Pressing down on the lid may permit the lid to engage by counteracting the bias enough to permit a buckle coupled to the switch to engage with the lid, as described in greater detail below. In alternate embodiments, a user may manipulate the switch to keep the lid in the closed position. When the user moves the switch backwards, the lid may be released and may open automatically.

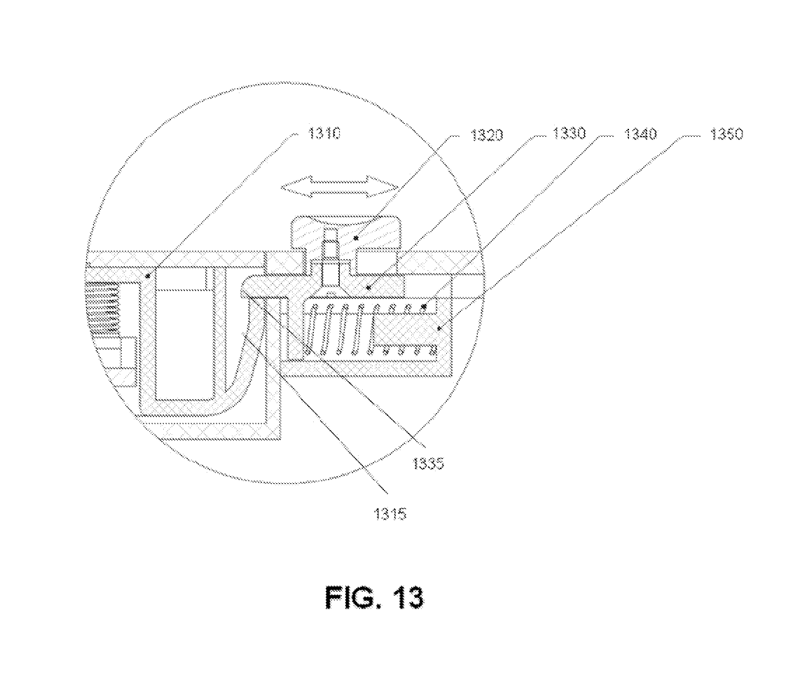

(4) When the lid is pressed down by the user, a bottom panel may compress the buckle to make it move towards the opposite direction until it reaches a notch/indentation. FIG. 13 shows a close up view of a lid release mechanism in accordance with an embodiment of the invention. A bottom panel 1310 of a lid may include a hooked portion 1315. The hooked portion may encounter a protruding portion 1335 of a buckle 1330. The protruding portion of the buckle may overlie the hooked portion of the bottom panel. This may prevent the bottom panel from moving upwards. A switch 1320 may be moved as indicated. Moving a switch backwards may cause the buckle to move backwards correspondingly. This may cause the protruding portion of the buckle to withdraw. When the protruding portion of the buckle no longer overlies the hooked portion of the bottom panel, the lid may open. A user moving the switch backwards may counteract a force exerted on the buckle by a switch spring 1340 and switch spring compression plate 1350 that may support the switch spring. When the lid is pressed down by the user, the switch and buckle may be pushed by the spring forward. When the lid comes down, the curved hook portion 1315 of the bottom panel may come over the protruding portion 1335 of the buckle. The curvature of the hook portion may cause the protruding portion of the buckle, and the rest of the buckle to be pushed backwards, when the hook portion is pressed down. The buckle may be pressed backwards against the switch spring until the protruding portion of the buckle comes over the curved hook portion of the bottom panel. This may cause the buckle to move back forwards due the spring force, and since the curved hook portion is no longer holding the protruding portion of the buckle back.

(5) Under the effect of the compression spring 1340, the buckle 1330 may lock the heater component. Thus, the lid of the lid assembly may remain closed until the user pushes the switch 1320 back. The lid may optionally remain closed even if the device is jiggled or re-oriented.

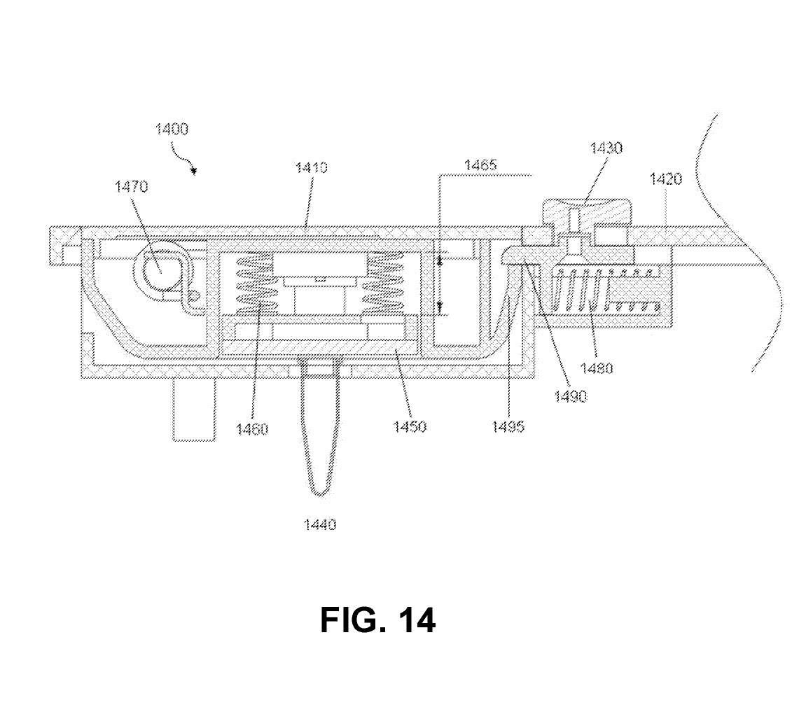

FIG. 14 shows a cross-sectional view of a lid configuration with adjustable portion in accordance with an embodiment of the invention. The lid may be configured to accommodate sample containers of different types. The lid may include a heater. It may be desirable for the lid to be opened and closed above the sample containers without manually adjusting the heater. In some embodiments, the heater height may automatically adjust to accommodate the different sample containers. The height of the heater may change depending on the type of tube.

A lid assembly 1400 may include a lid panel 1410 that may be capable of moving between an opened and closed position. The lid panel may form an outer surface of the lid assembly. The lid panel may be in line with an instrument panel 1420 or any other portion or surface of a device. A switch 1430 may be provided that may be moved backwards to cause the lid to open. The switch may normally be pressed forward using a force inducing component, such as a spring 1480 against a buckle coupled to the switch. The buckle may include a protruding portion 1490 that may overlie a hooked portion 1495 of a bottom panel of the lid. The bottom panel of the lid may be attached to the lid panel and/or may move with the lid panel. For example, when the lid panel moves between the open and closed position, the bottom panel may move correspondingly between the open and closed position. The lid panel may pivot about a shaft 1470 to move between the open and closed position. Optionally, a torsion spring may be provided around the shaft which may bias the lid panel to an open position. The protruding portion 1490 overlying the hook 1495 of the bottom panel may keep the lid closed. When the switch moves the protruding portion away from the hook, the bias of the torsion spring may come into effect, and the lid may open of its own accord. A user may press the lid closed to cause the protruding portion to re-engage with the hook and keep the lid closed.

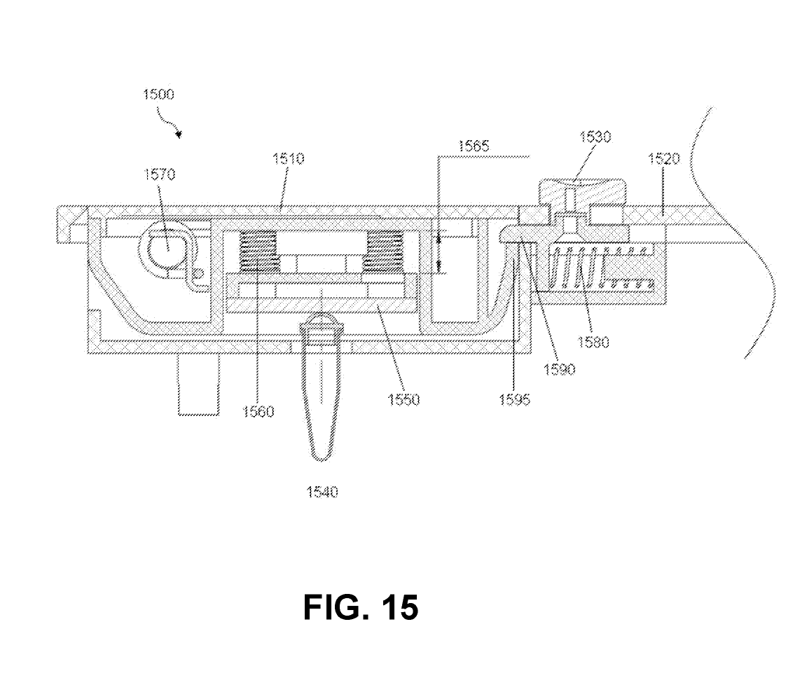

The lid may include a heated plate 1450. The heated plate may move with the lid panel 1410 and/or bottom panel. For example, when the lid panel is in an open position, the heated plate may move with the lid and be exposed. The temperature of the heated plate may be controlled. In some instances, the temperature of the heated plate may be controlled to any degree as described elsewhere herein for temperature control. The heated plate may be used for heating and/or cooling. The temperature of the heated plate may be controlled to within 0.01 degrees, 0.05 degrees, 0.1 degrees, 0.5 degrees, 1 degree, 2 degrees, 3 degrees, 4 degrees, 5 degrees, 7 degrees, or 10 degrees Celsius. The heated plate may come into contact with a sample container 1440. The sample container may be configured to contain sample therein. The sample container may have any configuration. For example, a flat top sample container may be provided. Alternatively, as illustrated in FIG. 15, a dome-topped sample container may be provided. The heated plate may contact the top of the sample container. The heated plate may heat and/or cool the sample container and/or sample contained therein. One or more force inducing components, such as springs 1460 may be provided that may provide flexibility in the height of the heated plate. The springs may bias the heater downward. The springs may cause the heater to be pressed against the top of the sample container. The springs may ensure good contact between the heater and the sample container top. A spring displacement 1465 may be provided.

FIG. 15 shows a cross-sectional view of a lid configuration with adjustable portion in accordance with an embodiment of the invention. As previously described, a lid assembly 1500, may have a lid panel 1510, instrument panel 1520, switch 1530, switch spring 1580, buckle with protruding component 1590, bottom panel with hook portion 1595, shaft 1570, heater 1550, and/or heater springs 1560.

The heater 1550 may be configured to contact the top of a sample container 1540. In some instances, the sample container may be dome topped sample container. The sample container may have a greater height than a flat-topped sample container 1440. The springs 1560 may bias the heater against the top of the sample container. A spring displacement 1565 may be provided.

When the height of a first sample container 1540 is greater than the height of a second sample container 1440, the spring displacement for the first set-up 1565 may be less than the spring displacement for the second set-up 1465. In some instances, a height may be provided between a bottom surface of a bottom panel and a top surface of a supporting member 1545 that may support the sample container. The height may be fixed. When the sample container top pushes the heater higher up away from the supporting member, a smaller spring displacement may be provided.

The sample container may have any height that extends beyond the supporting member 1545 top surface. For example, the sample container may have a height of less than about 0.1 mm, 0.5 mm, 1 mm, 1.5 mm, 2 mm, 3 mm, 4 mm, 5 mm, 6 mm, 7 mm, 8 mm, 9 mm, 1 cm, 1.1 cm, 1.2 cm, 1.3 cm, 1.4 cm, 1.5 cm, 1.7 cm, 2 cm, 2.5 cm, or 3 cm, extending beyond the upper surface of the supporting member. The sample container may have a height extending beyond the top surface of the supporting member greater than any of the height values mentioned herein. The sample container may a height extending beyond the top surface of the supporting member within a range between any two of the values described herein. The heater may be capable of accommodating the sample containers having any of the height values mentioned herein. The heater may be capable of accommodating a wide range of heights. For instance, the heater may contact tops of sample containers, over a range of greater than or equal to 0.1 mm, 0.3 mm, 0.5 mm, 0.7 mm, 1 mm, 1.5 mm, 2 mm, 2.5 mm, 3 mm, 4 mm, 5 mm, 7 mm, 1 cm, 1.5 cm, 2 cm, 2.5 cm, 3 cm, 3.5 cm, 4 cm, 4.5 cm, or 5 cm. In some instances, the heater may be capable of accommodating 0.1 mL, 0.2 mL, or 0.5 mL sample containers with flat tops and/or dome tops. The heater may be able may be able to account for variations in height that are greater than 1%, 3%, 5%, 10%, 15%, 20%, 25%, 30%, 40%, or 50% of the height of the sample containers.

The heater may be compressed different amounts depending on the height of the sample containers. The heater may be compressed different amounts depending on the shape of the top surface of the sample containers (e.g., flat top vs. dome top). The heater may contact the tops of the sample containers regardless of the various configurations described. When the tops of the containers within the device at a given moment in time are the same height, the heater may contact the tops with substantially the same amount of force (e.g., with a force variation of less than or equal to 1%, 3%, 5%, 10%, 15%, 20%, 25%, 30%, 40%, 50%, 60%, 70%, or 80%. The heater may remain substantially parallel relative to the heater panel. The heater may remain substantially parallel relative to the heater panel regardless of the degree of compression. Alternatively, there may be some variation in the angle of the heater with respect to the heater panel.

The heater may be capable of providing any range of spring displacement. For example, the heater may be capable of providing less than a 1 mm, 3 mm, 5 mm, 7 mm, 1 cm, 1.5 cm, 2 cm, 2.5 cm, 3 cm, 3.5 cm, 4 cm, 4.5 cm, 5 cm, 6 cm, 7 cm, or 8 cm range of spring displacement. The heater may be capable of providing a range of spring displacement greater than any of the values described herein. The heater may be capable of providing a range of spring displacement falling between any two of the values described herein.

The heater of the lid may be configured to accommodate various sample container configurations. This may be done without requiring any electrical signals to be sent, or electrically controlled components. In some instances, the heater may accommodate the various sample container configurations using mechanical configurations. For instance, a force-inducing component, such as compression springs may be utilized. Other types of force inducing components, such as elastics, tension bands, compressible materials, bubbles forms, balloons, or bladders may be used.

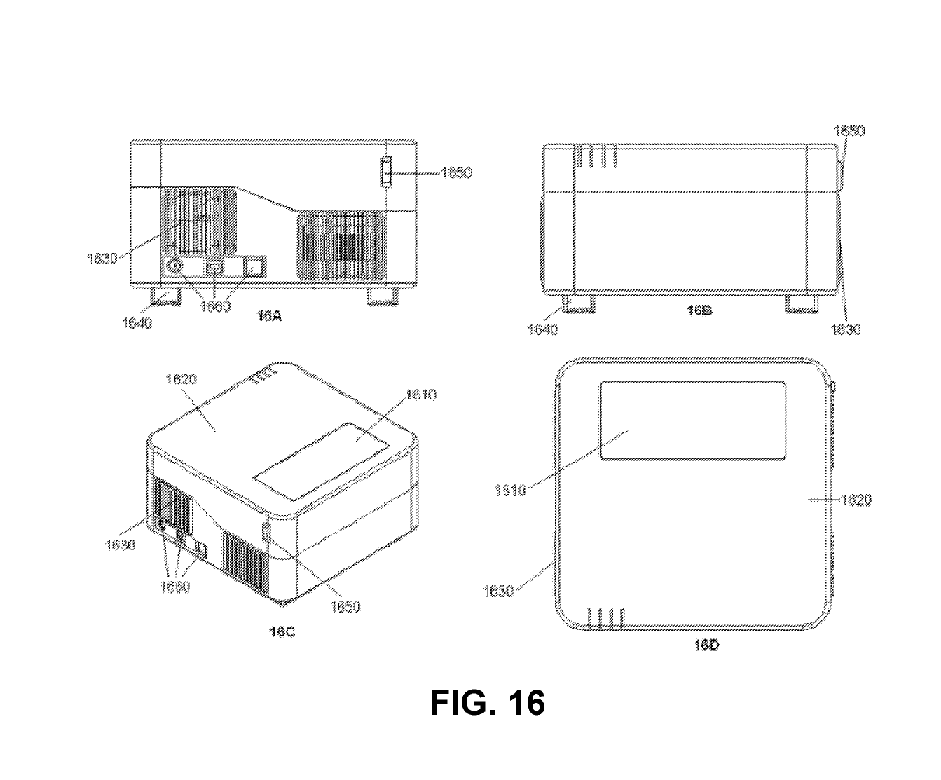

FIG. 16 shows multiple views of a device having a lid in accordance with an embodiment of the invention. In some embodiments, the device may be nucleic acid amplification device. The device may be any other type of device. The device may include a lid 1610. A heater may optionally be provided in the lid. The rest of the housing 1620 of the device may be provided. The housing may enclose one or more portions of the device. The housing may include vents 1630 that may aid with heat exchange with the ambient environment. One, two, or more fans may be provided that may dissipate heat. One or more fans may dissipate heat for a heat sink working for a temperature control unit, and one or more fans may dissipate heat for heating elements in a circuit control area. The fans may aid in ensuring that the device may be adaptable to temperature change. In some embodiments, a lid assembly may have a separable heat source and lid. This may reduce the impact of heat on a lid panel material and/or may also improve safety of operation.

Optionally one or more stands 1640 may be provided, which may elevate a bottom surface of the device. This may aid in heat exchange with the ambient environment.

A switch 1650 may be provided. The switch may be used to turn the device on or off or control one or more functions of the device. The switch may be used to open the lid of the device. The switch may be used for controlling opening and/or closing of the lid 1610 of the device.

One or more connection ports 1660 may be provided. The connection ports may be used to connect the device to one or more other devices and/or a power source. The connection port may be a power connection port, a data connection port, or any other type of connection port. The device may be connected to a power source, an energy storage device such as a battery pack, a computer or other device, a display device, or any other type of device. In some instances, a power input operation panel may be provided.

In some embodiments, the device may have multiple compartments. For example, two compartments may be provided, such an upper and lower compartment. The upper and lower compartments may form parts of the housing. The upper and lower compartments and/or portions of the housing may be separable from one another.

A working zone may be located in the upper part. An indicator light may be provided. The indicator light may provide information about the status of the device. The indicator light may indicate whether the device is in operation.

The device may have the overall dimensions of about 195 mm×195 mm×112 mm. The device may have any other dimensions as described in greater detail elsewhere herein. The device may be portable. The lid portion of the device may be dimensions to fit on a portable device.

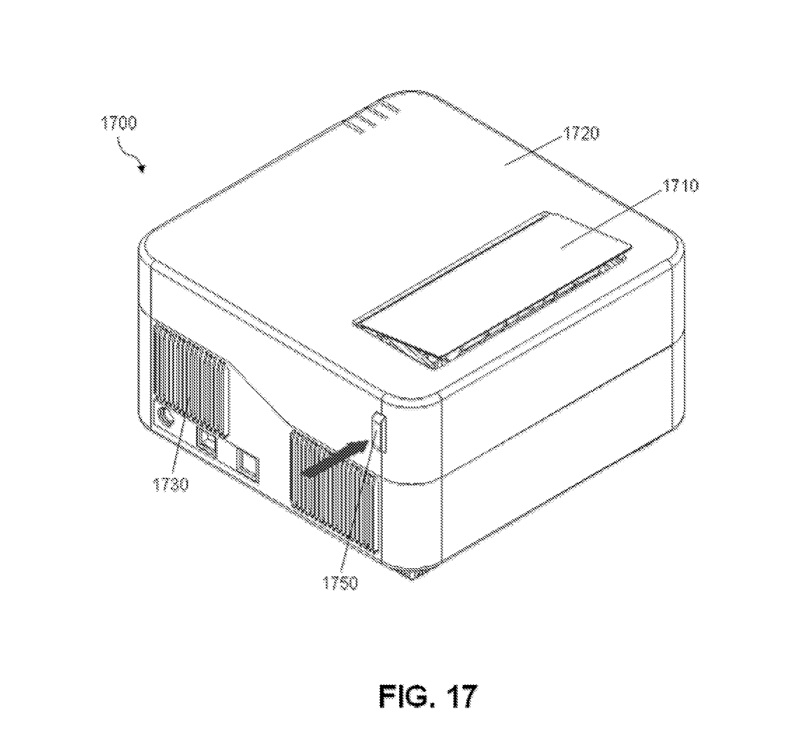

FIG. 17 shows a view of a device 1700 with a lid 1710 in a partially opened position in accordance with an embodiment of the invention. The lid may move relative to a housing 1720 of the device. The lid may move by rotating about one or more axes. The lid may rotate about a shaft. The orientation of the lid may change as the lid opens.

As previously described one or more vent or heat exchange component 1730 may be provided.

A switch 1750 may be provided. Optionally, the switch may be used to control operation of the lid 1710. The switch may be moved to cause the lid to open. Pressing the switch may cause a release of the lid. In some instances, the switch may be used to cause the lid the close. In other instances, a user may close the lid by manually pressing down on the lid. As previously described, the switch may be a lid-releasing mechanism. Manually manipulating the lid-releasing mechanism may cause the lid to open without further interference from the user.

The switch may be located on any portion of the device. The switch may be located on a same surface of the device as the lid. For example, if the lid is on a top surface of the device, the switch may also be located on a top surface of the device. Alternatively, the switch may be located on a different surface of the device as the lid. For instance, the lid may be on the top surface of the device while the switch may be a side surface of the device. The switch may move laterally with respect to the lid, or may move vertically with respect to the lid.

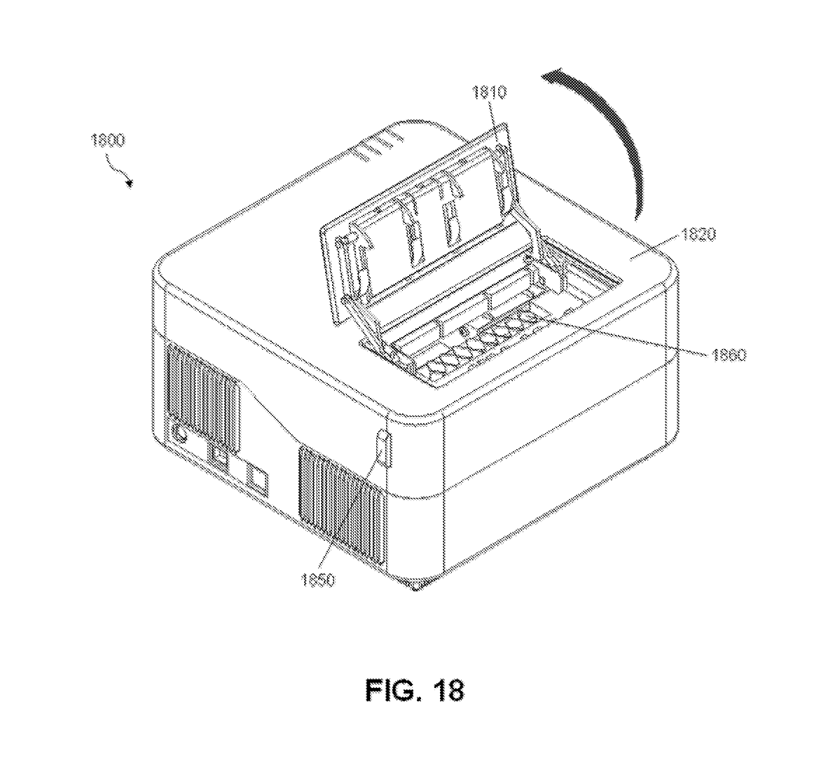

FIG. 18 shows a view of a device 1800 with a lid 1810 in an open position in accordance with an embodiment of the invention. The lid may be open relative to the rest of the housing 1820. The switch 1850 may have been pressed to cause the lid to open. Any description of pressing the switch may apply to any type of manipulation of a lid-releasing mechanism. One or more sample containers 1860 may be accessible when the lid is opened. A sample container may be inserted into the device or removed from the device when the lid is open. The top of the sample container may be viewable when the lid is opened.

Optionally, a heater may not be viewable when the lid is opened. The heater may optionally not be provided on the bottom of the lid when the lid is opened.

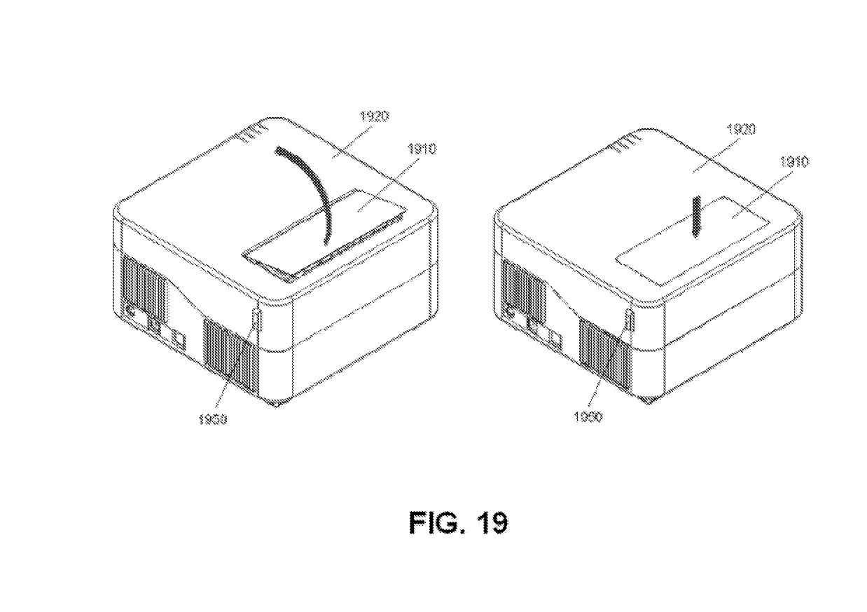

FIG. 19 shows views of a device with a lid 1910 being closed in accordance with an embodiment of the invention. After an operation is completed (e.g., adding or removing a sample container, or viewing a sample container), the lid may be closed. The lid may be manually pressed downward to be in line with the rest of the housing 1920. In some instances, the lid may be pressed tight to cause an internal latching mechanism to engage. After the lid is pressed closed, a user may engage a switch 1950 in order to open the lid again.

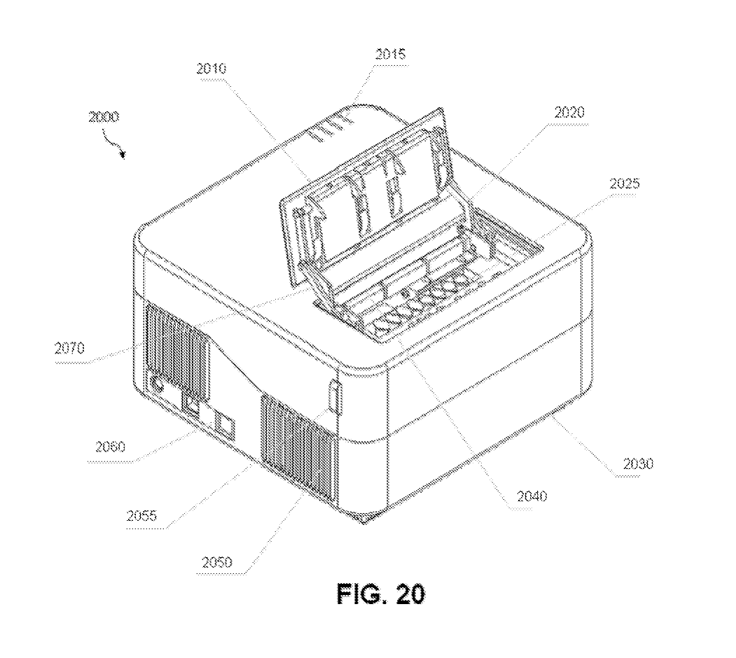

FIG. 20 shows a device 2000 with a lid 2010 in an open position with a retractable portion 2040 in accordance with an embodiment of the invention. The retractable portion may be a heater. The heater may be used to heat and/or cool one or more sample containers 2025 therein. When the lid is closed, the heater may contact the sample containers. When the lid is closed, the heater may press against the top of the sample containers. When the lid is closed, the sample containers may be covered by both the heater and the lid. When the lid is closed, the heater may be located between the sample containers and the lid. When the lid is closed the heater and/or the lid may be substantially parallel to one another (e.g., within less than or equal to 1 degree, 3 degrees, 5 degrees, 10 degrees, 15 degrees, 20 degrees, or 30 degrees of one another).

When the lid is opened, the heater may retract. The lid and the heater may be capable of moving along independent paths. For example, the lid and the heater may have different paths between the closed and open positions. The lid may change orientation between the closed and open positions. The lid may rotate about an axis of rotation between the closed and open positions. The lid may follow a curved path between the closed and open positions. The heater may optionally not change orientation between the closed and open positions. The heater may translate in a direction without rotation or substantial rotation between the closed and open positions. The heater may follow a straight path between the closed and open positions. The heater may move in a linear direction. The heater may move horizontally. The heater may optionally have little or no vertical movement (e.g., less than 5 degrees of vertical movement). The heater may optionally retain the same orientation between the closed and open position. When the lid is opened the heater and/or the lid may end up at different angles relative to one another (e.g., at greater than or equal 45 degrees, 50 degrees, 60 degrees, 70 degrees, 80 degrees, or 90 degrees relative to one another). The heater may retract into the device. The heater may retract so that it is within the housing/case 2030 of the device. The heater may be under a surface of the housing of the device when the lid is opened. While the lid may be rotating into an open position, the heater may be sliding laterally to move under the housing of the device. This may permit the sample containers to be exposed to the user when the lid is opened. The movement of the lid may drive the movement of the heater. The speed at which the heater retracts may depend on the speed at which the lid is rotating. The position of the heater along its lateral path may depend on the position of the lid along its rotational path. The lid and heater may be connected to one another with mechanical connection, without requiring electrical signals or components to drive the motion of the lid and/or heater. The movement of the lid may cause the movement of the heater via a mechanical coupling. The heater may retract so that when a lid is opened, a heater is not exposed to the user. The heater may be retracted so that when the lid is opened, the heater is substantially hidden from the user. This may prevent the user from accidentally coming into contact with the heater when the lid is opened. This may prevent the user from being burned by the heater if any heat or residual heat is provided on the heater.

One or more connecting rods may be provided for the lid 2010 and/or heater 2040. A first connecting rod 2020 may be used to connect the lid with a shaft, or other component passing through the axis of rotation for the lid. The first connecting rod may optionally be curved or bent. A second connecting rod 2070 may be used to connect the lid with a slider that may control movement of the heater. The second connecting rod may optionally be straight. The connecting rods may be configured so that the movement of the lid will drive the movement of the retractable heater. The rotation of the lid may effect the translation of the retractable heater. The movements of the lid and the retractable heater may occur simultaneously.

The device may have a housing/case 2030. One or more indicator lights 2015 may be provided. The indicator lights may be indicative of a status of the device (e.g., on or off, undergoing thermal cycling or not, etc.). A switch 2050 may be provided which may be used to release the lid. Pressing the switch may cause the lid to open and/or the heater to retract. One or more vents 2050 and/or connection ports (e.g., input panel) 2060 may also be provided.

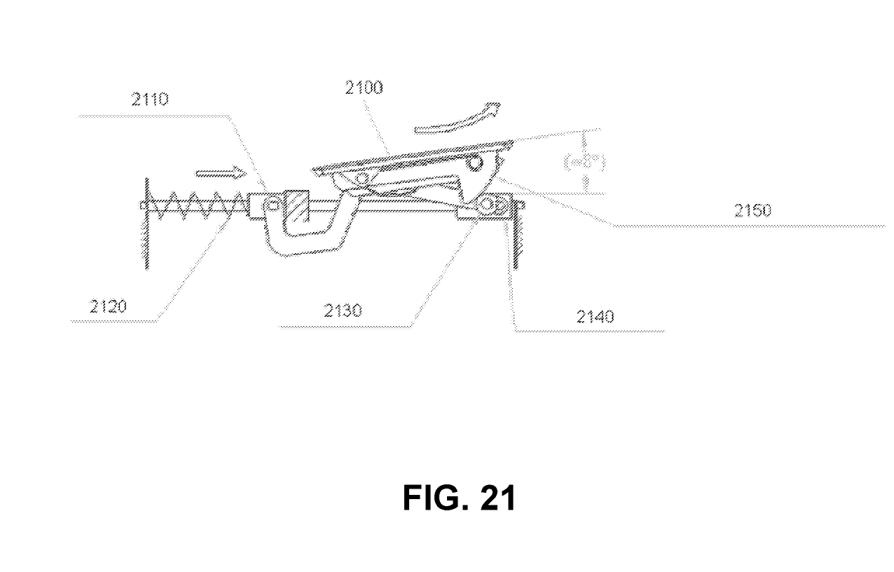

FIG. 21 shows a lid apparatus in a partially opened position in accordance with an embodiment of the invention. A lid opening/closing mechanism may be provided. A lid apparatus may be opened by pressing a switch button, which may cause the manual release and opening of the lid. Any other type of lid-releasing mechanism may be employed. The opening experience may provide a pleasing audio and/or tactile experience. Sound from an additional spring system may contribute to the pleasing experience for the user. When the switch is pressed, one or multiple turning rolls may be moved simultaneously. The turning of multiple rolls may ensure that uniform force is applied. This may be convenient and labor efficient.

A lid panel 2100 may be capable of opening and closing. The lid panel may be connected to a slider 2110. A force inducing component, such as a spring 2120 may press against the slider 2110. The spring may cause the slider to be held against a stop/limit in a forward position. In some embodiments, the spring may be held back (e.g., when the lid is closed). The spring may be released when the lid is opened and/or when a switch is depressed to open the lid. The spring may move the slider up to the limit, which may cause the lid to move at an angle and partially open. Once the lid starts to open, the slider may remain in a substantially fixed position when pressed by the spring. When a user presses down on the lid to close the lid, the slider may move in a limited fashion.

A turning roll 2130 may be provided. The turning roll may have a roller shaft fixed. Any other type of mechanism may be employed that may cause a similar motion to the turning roll. A second slider 2140 may be provided. The second slider may move along a slider guide. The second slider may be connected to the lid via a connecting rod. The movement of the lid may cause the movement of the second slider. The second slider may slide along a wide range along the slider guide. The hook for the lid may be released, which may cause the lid to open 2150. In some instances, selecting a switch may cause the lid to be released, which may cause the lid to slide forward and rotate slightly, due to the spring 2120 pressing on the first slider 2110. The slight opening of the lid may or may not cause movement of the second slider and the retractable heater.

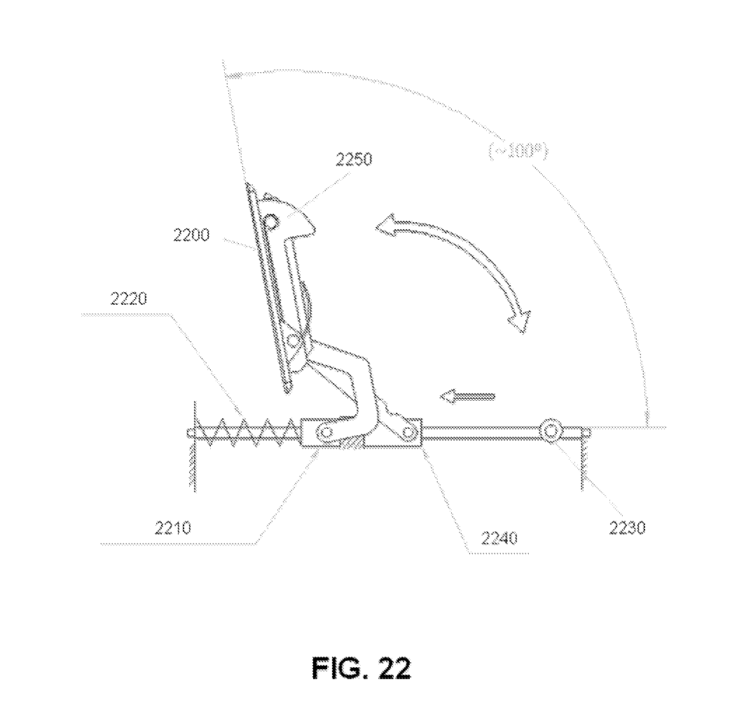

FIG. 22 shows a lid apparatus in an open position in accordance with an embodiment of the invention. The lid apparatus may be opened after the switch has been used to release the lid. The lid may be manually opened. The lid may open to a fully opened position with aid of a torsion spring or similar force inducing component that may cause the lid to rotate. In other implementations, actuators, such as motors or other components may be employed. In some examples, the lid may be manually opened using a user's hand.

A lid panel 2200 may be opened. The lid panel may rotate and/or re-orient to reach a fully opened position. A maximum open position may be attained. The maximum open position may have any degree value, such as less than or equal to about 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, or 10 degrees. A spring 2220 or other force inducing component may be released, which may cause a first slider 2210 to be provided at a limit fixed location. A second slider 2240 may slide along a slide guide. The second slider may slide in response to movement of the lid panel. The second slider may be connected to a lid, so that the movement of the lid, drags the slider to slide along the slide guide. When the lid is opened further, the slider may move backwards (e.g., toward a hinge or rotational axis of the lid). A roller 2230 may be provided.

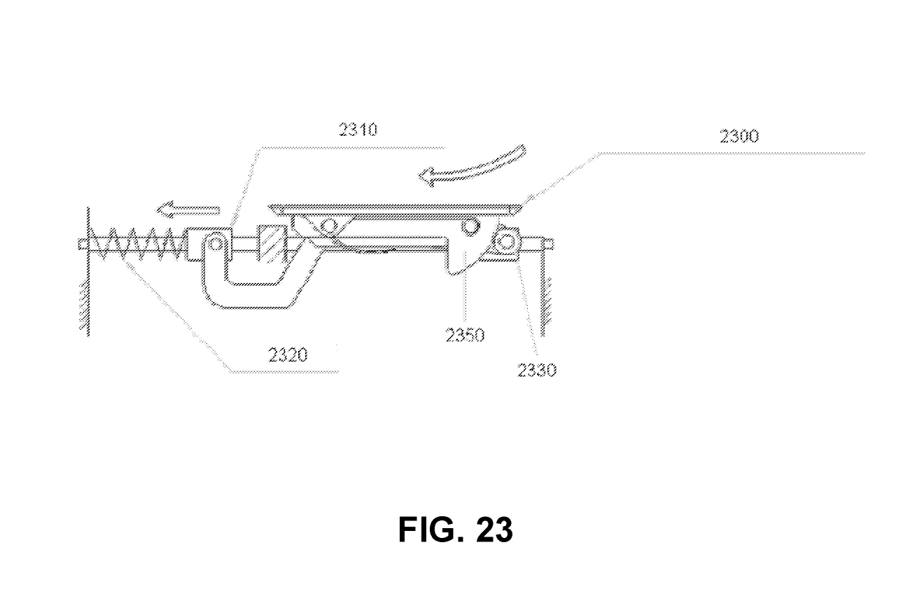

FIG. 23 shows a lid apparatus in a closed position in accordance with an embodiment of the invention. A user may press the lid to return the lid to a closed state. The lid panel 2300 may be pressed down. A hook limit may be locked. For example, the lid panel may be hooked closed so that it may not open without the hook first being released. A first slider 2310 may be disengaged from the limit stop. The first slider may move backwards when the lid is closed. The first slider may be counteracting a force induced by a component, such as a spring 2320. The spring may be compressed. A roller portion 2330 may be provided. A hook shaped portion 2350 may be provided.

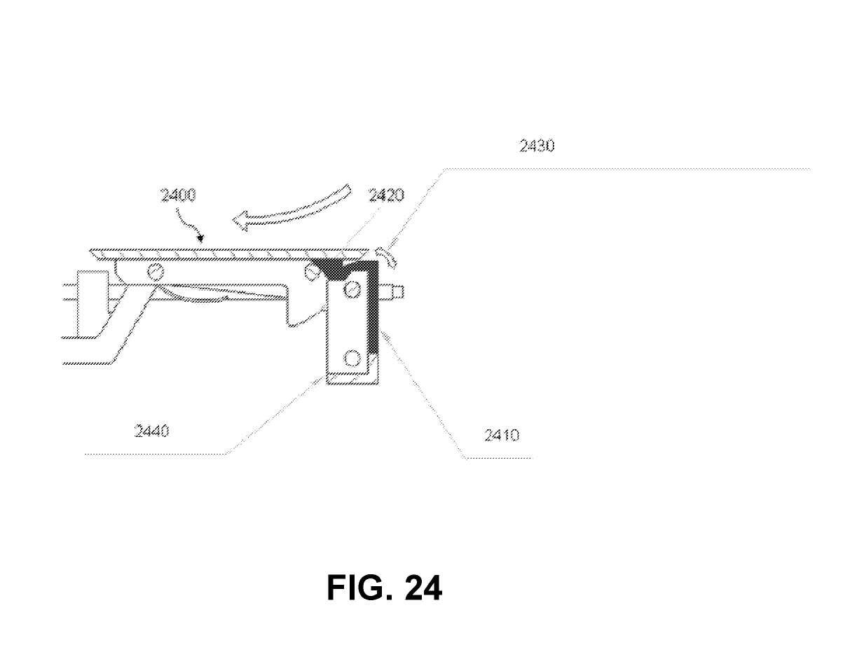

FIG. 24 shows a locking mechanism for a lid apparatus in accordance with an embodiment of the invention. An elastic hook group may be adopted. Upon locking, one, two, three, four, five or more hooks may function simultaneously to ensure reliability of the limit. The hooks can aid in the opening operation of a switch button.

A lid panel 2400 may be provided. The lid panel may include a hooked portion 2420. The hooked portion of the lid panel may move with the lid. The hooked portion of the lid panel may be affixed to the lid panel. A complementary hook 2410 may be provided. The complementary hook may be part of an elastic hook group 2440. The elastic hook group may include any number of hooks that may engage with a hook portion of the lid.

When the lid is pressed down, the hook 2410 may be slightly opened while the hooked portion 2420 of the lid is pressing on it. The hook 2410 may rebound when a locking status is reached (e.g., when the hooked portion 2420 falls under the hook 2410). Then when a lid is pressed completely down, the hooks may engage and latch closed. A click or other confirmation of locking may occur.

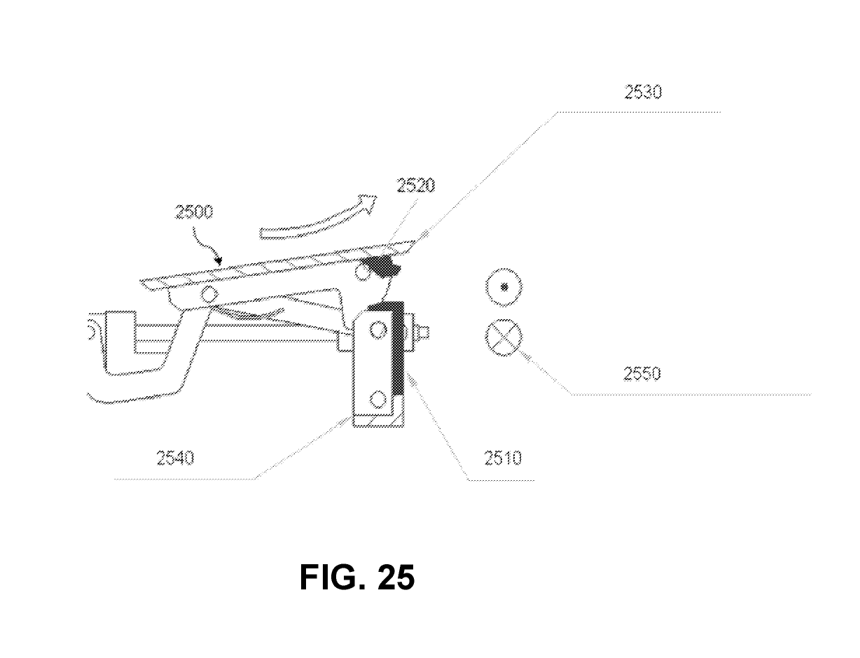

FIG. 25 shows a lid apparatus where the locking mechanism has been released in accordance with an embodiment of the invention. When the lid apparatus is in an unlocked state, the lid panel 2500 may be partially opened. The hooked portion 2520 of the lid panel may be disengaged from a complementary hook 2510. The complementary hook may be part of an elastic hook group 2540. In one example, an elastic hook group may connect and support a plurality of hooks. The disengagement of the hooked portions may be indicative that the lid is unlocked and released 2530. A switch button 2550 may be driven to cause an elastic hook group to move vertically to unlock the lid.

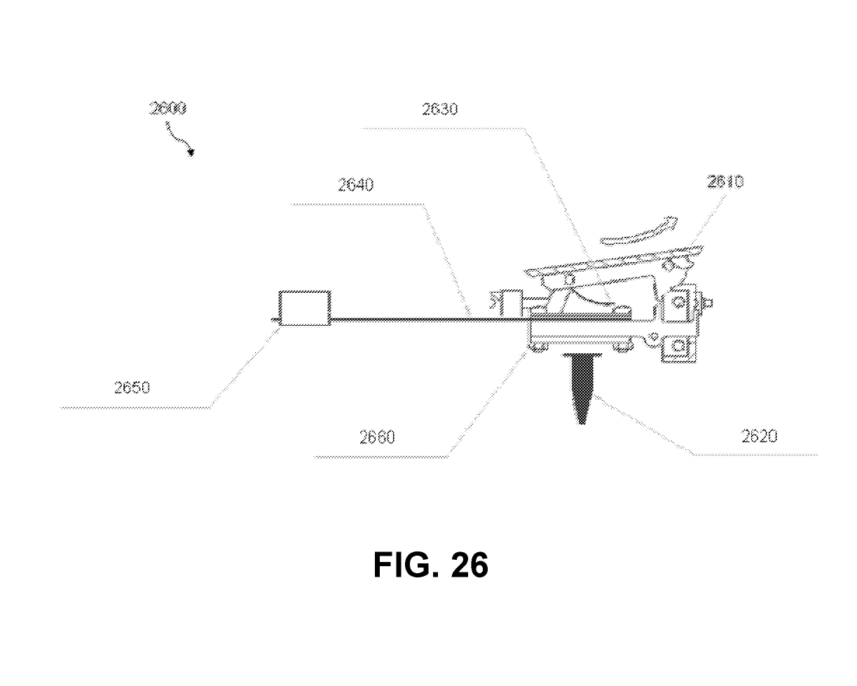

FIG. 26 shows a lid apparatus being opened in accordance with an embodiment of the invention. The lid apparatus 2600 may include a lid panel 2610. The lid panel may open and close to expose and cover one or more sample containers 2620. When the lid panel is opened, the sample containers may be exposed and/or accessible from outside a device. In some instances, when the lid is initially opened, the lid may partially lift from the surface of the device. Optionally, the lid may move forward slightly in a direction opposite of the direction in which the heater will retract. When the lid panel is closed, the sample containers may be inaccessible from outside the device.

The lid panel 2610 may have a force inducing component 2630. The force inducing component may be used to press down on the sample container 2620. The force inducing component may ensure that the sample container is pressed down even after the lid is closed. This may prevent or reduce the likelihood of spilling reagents within the container upon heating. In some instances, the force inducing component may include a leaf spring or other type of spring. Any number of force inducing components may be provided. For example, one or more, two or more, three or more, four or more, five or more, or six or more leaf springs in the lid can exert a downward force. The downward force may be exerted directly on the sample container or may be exerted on a heat tongue 2660 which may transfer the force to the sample containers. A steel wire 2640 may be connected to the heat tong. A guiding block 2650 may be connected to the steel wire. The guiding block may have a fixed position. FIG. 26 shows the steel wire, heat tongue, force inducing component and/or sample container in a free state. The free state may be provided when the force inducing component is not exerting a force (e.g., when the lid is not pressed down).

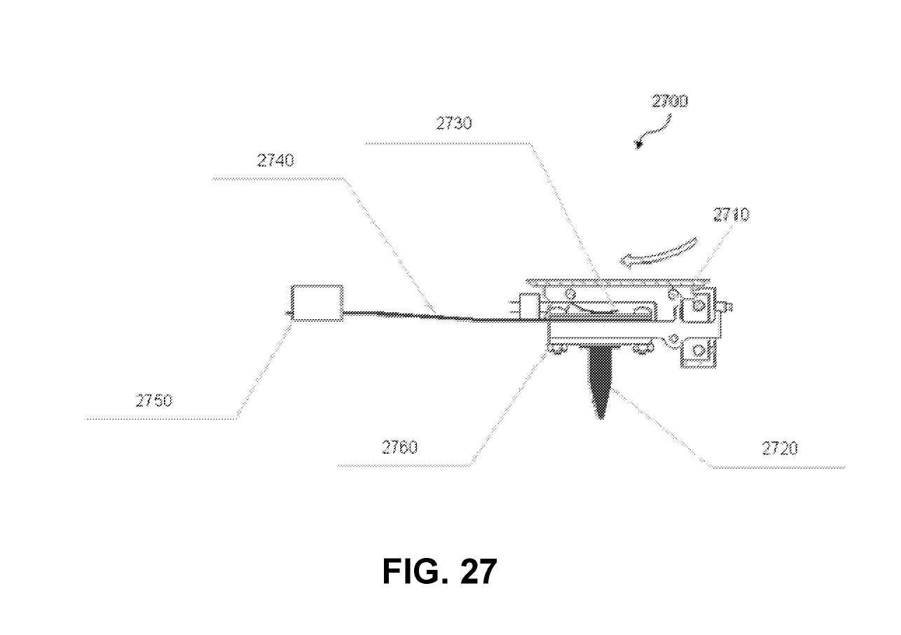

FIG. 27 shows a lid apparatus 2700 being closed in accordance with an embodiment of the invention. When the lid panel 2710 is pressed down, a heat tongue 2760 may descend and press on a sample container 2720. One or more force inducing components 2730 such as leaf springs of the lid may press down on the heat tongue when the lid is pressed down. The force inducing components may cause the heat tongue to be pressed against the sample container. The force inducing component may permit the heat tongue to accommodate different heights of sample containers. For example, the force inducing component may permit the heat tongue to press against flat topped sample containers and/or dome topped sample containers. The force inducing component may permit the heat tongue to accommodate sample containers having varying heights above a supporting element, such as any of the height values mentioned elsewhere herein. The heat tongue may be heater that may be used to heat and/or cool the sample containers. The heat tongue may be pressed against the sample container to provide good thermal contact.

When the heat tongue 2760 presses down on the sample container, the guide wire 2740 may be bent under the force. One end of the guide wire may be connected to a fixed guiding block 2750. The guide wire may be formed from a metal or metal alloy, such as steel. FIG. 27 shows the steel wire, heat tongue, force inducing component and/or sample container in a pressed down state. The pressed down state may be provided when the force inducing component is exerting a force (e.g., when the lid is not pressed down).

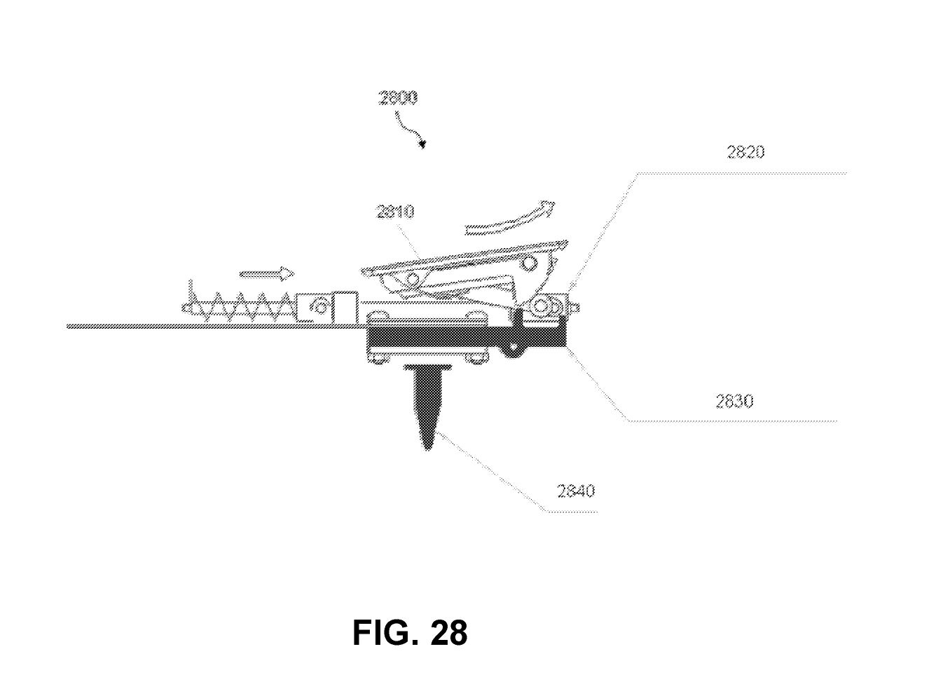

FIG. 28 shows an additional view of a lid apparatus 2800 being opened in accordance with an embodiment of the invention. A retractable heater (a.k.a. heat tongue) 2830 may move as a lid 2810 is opened and/or closed. When the lid moves between an open and closed position, the heater may move between a retracted and extended position. When the lid is in an open position, the heater may be retracted and may not cover a sample container 2840. Thus, when a lid is in a fully open position, the sample container may be uncovered and accessible by a user. When the lid is in a closed position, the heater may be extended and may cover the sample container. Thus, when the lid is in a fully closed position, the sample container may be covered by both the heater and the lid. The sample container may not be accessible by the user. A lid may be in a partially opened and/or partially closed state. The heater may be partially retracted and/or extended in those states.

In one instance, a lid may be provided in a closed position, in which the exterior surface of the lid may be in line with an exterior surface of the device. The sample container may be covered by the lid and the heater. The lid may be moved to a partially open position (e.g., free state). The top surface of the lid may extend higher than the exterior surface of the device. The lid may move slightly forward (e.g., opposite direction as the direction in which the heater will retract). The orientation of the lid may change slightly from a fully closed to partially open state (e.g., less than 15 degrees, 10 degrees, 8 degrees, 6 degrees, 5 degrees, 4 degrees, 3 degrees, 2 degrees, or 1 degree). When the lid is in the initial partially open state, the heater may optionally still cover the sample container. The lid may move through intermediary positions toward a fully open state. While the lid is rotating about the intermediary positions, the heater may be retracting, to uncover the sample container. The lid may rotate upwards and rearwards to a fully open position. The heater may retract rearwards linearly to the fully open position.

A guiding slider 2820 may be provided. The guiding slider may connect to a lid via a connecting rod. The slider may pivot with respect to the connecting rod. The angle of the connecting rod relative to the slider may change. The guiding slider may connect to a heater. A slider pin may be provided to permit the slider to get in touch with the heater.

The heater 2830 may be a separable heat source from the lid 2810. Having the separable heater may reduce the impact of heat on the lid panel material and may also improve safety. Design of the heater with the directed guide wires may cover a dual function of a guide rail spring, which can be simple and low cost. Also, the driving force of the heater movement may come from the opening and/or closing of the lid by the user. This may provide a reliable and energy efficient way of effecting movement of the heater. Thus, the heater may be retracted and/or extended in response to mechanical movement. No electricity may be required to effect the movement of the heater.

FIG. 28 shows the lid assembly in a free state. Thus, the lid panel may be released, the heater may be relaxed, and the sample container may be free. In the free state, the sample container may still be covered by the heater.

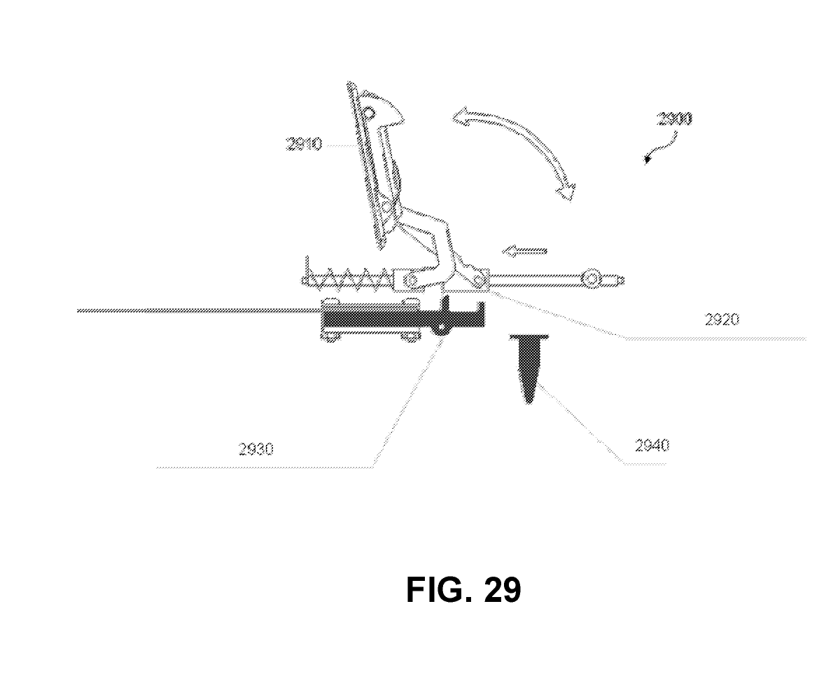

FIG. 29 shows an additional view of a lid apparatus 2900 in an open position in accordance with an embodiment of the invention. The lid panel 2910 may be opened. A slider 2920 may be moved backwards in response to the opening of the lid panel. The slider movement may be driven by the movement of the lid panel. The slider movement may be limited one or more guide rails. The retractable heater 2930 may be similarly retracted and moved backwards in response to the opening of the lid panel. The retractable heater may be coupled to the slider and may move backwards with the slider. A sample container 2940, such as a test tube, may be uncovered.

The lid panel 2910 may open and/or close, and the heater 2930 may be driven and may induce shrinking and/or stretching of a guide wire, such as a steel wire.

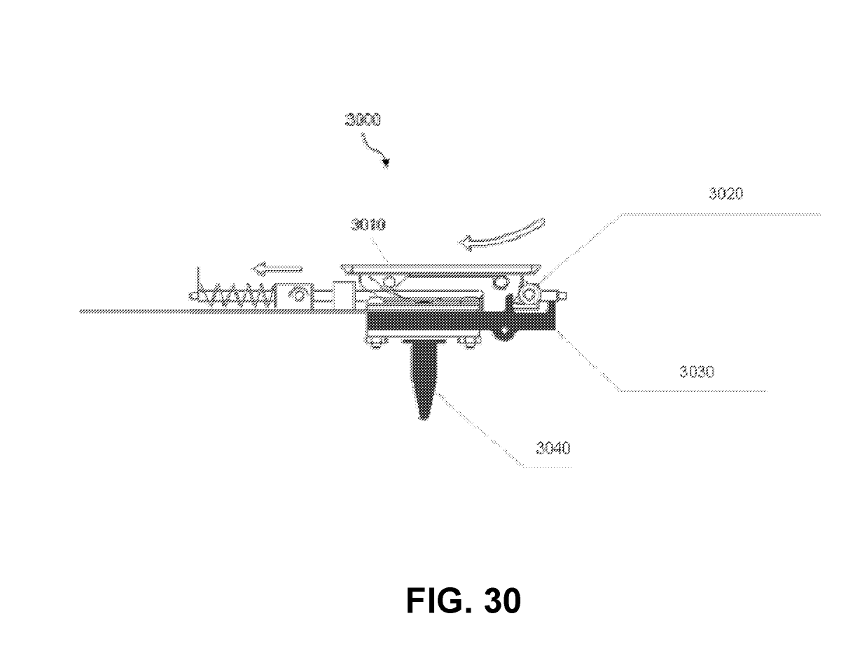

FIG. 30 shows an additional view of a lid apparatus 3000 being closed in accordance with an embodiment of the invention. A lid panel 1030 may be pressed down to be closed. A slider 3020 may be pressed forward when the lid panel is pressed down. The heater 3030 may be extended. The heater may be engaged with the slider and may be extended because the slider is positioned in a forward position. The heater may cover a sample container 3040.

The lid 3010 may be pressed tight, which may position the heater 3030 in place, and press down on the sample container 3040. The prep for the test may be done.

In some embodiments, the retractable portion may be a heater, and may be driven by the movement of the lid. The retractable heater may be separable from the lid and/or capable of moving along an independent path. In alternative embodiments, the lid may include a heater that may move with the lid. The retractable portion may be a thermally conductive metal portion that may come into contact with a heater that moves with the lid when the lid is pressed down. The thermally conductive retractable portion may conduct the heat from the heater of the lid to an underlying sample container. The lid may press the retractable portion on the sample container which may aid in forming good thermal contact.

The lid may have a heater as described herein. The heater may move with the lid, or may move in a different path from the lid. The heater may be configured as described in any of the various embodiments herein. The heater may be used to heat and/or cool a sample container when the lid is closed. The heater may or may not directly contact the sample container when the lid is closed. The heater may control the temperature of the top of the sample container. This may advantageously prevent fogging and/or condensation on the top of the sample container. This may permit signals to be detected more easily through the top of the sample container. For example, optical signals may pass through the sample container to be detectable by one or more detector. This may occur in real-time during the temperature cycles. The temperature of the lid heater may be controlled precisely (e.g., within less than 0.01 degrees, 0.05 degrees, 0.1 degrees, 0.5 degrees, 1 degree, 2 degrees, 3 degrees, 5 degrees, 7 degrees, or 10 degrees Celsius). The temperature of the lid heater may depend on a temperature of a supporting member within the device that may be used to heat and/or cool the sample. The temperature of the lid heater may match the temperature of the supporting member. The temperature of the lid heater may be within 0.1%, 0.5%, 1%, 3%, 5%, 7%, 10%, 15%, 20%, 25%, 30%, 40%, or 50% of the supporting heater. The temperature of the lid heater may remain less than the temperature of the supporting heater, greater than the temperature of the supporting heater, or substantially equal to the temperature of the supporting heater. When the temperature of the supporting heater rises, the lid heater temperature may also rise. When the temperature of the supporting heater decreases, the lid heater temperature may also decrease. In some instances, the temperature profile of the sample along the height of the sample container may remain substantially the same (e.g., vary by less than or equal to 0.1%, 0.5%, 1%, 3%, 5%, or 10%.

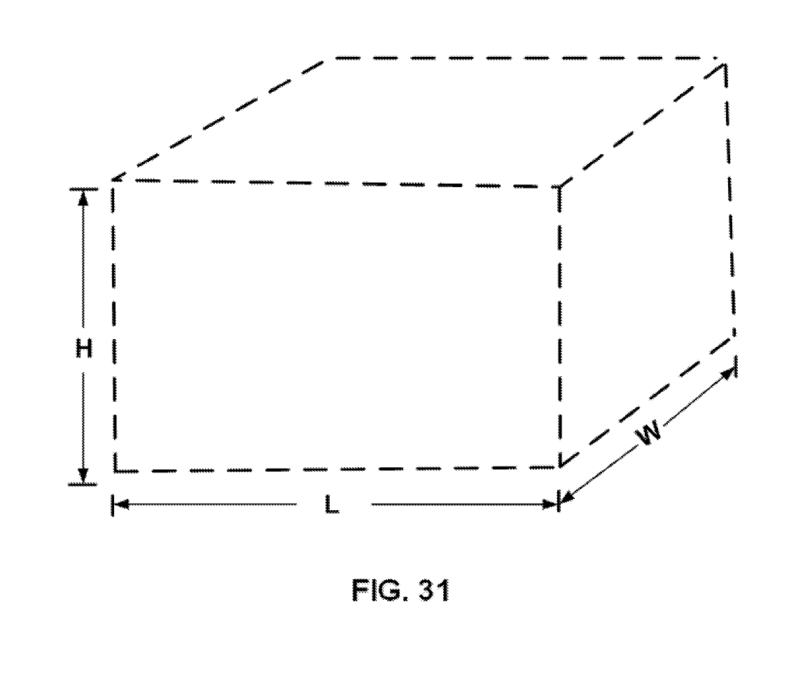

FIG. 31 shows an example of dimensions within which a device for conducting nucleic acid amplification may fall, in accordance with an embodiment of the invention. The device may be a portable device. The device may be capable of being lifted and carried by a human. The device may be capable of being lifted and carried by a human with one hand. The portable device may be capable of being transported via a passenger vehicle. A portable device may be desirable to deploy the device to various locations. The portable device may permit point of care (POC) nucleic acid amplification. This may permit individuals in remote areas to get faster results, which can be useful for disease prognosis and treatment.

The device may have a length L, height H, and/or width W. The device may have any shape. For example, the device may have substantially rectangular prismatic shape, rounded shape, triangular shape, hexagonal shape, cylindrical shape or any other shape. The device may fit within the dimensions illustrated even if the shape of the device does not cause the device to fill in the whole dimensions. The length may refer to the greatest lateral dimension of the device. The height may refer to the distance between the bottom and the highest point of the device. The width may refer to the dimension of the device in a direction orthogonal to the length. Any description herein of a dimension of the device may also refer to a dimension of a housing that may at least partially enclose one or more components of the device.

The device may have a maximum dimension (e.g., length, width, height, diagonal, diameter) of no more than about 15 cm. In some instances, the device may have a housing no more than 10 cm tall. In another example, the device may have a housing no more than 16 cm in length. The device may have a maximum dimension of no more than about 1 mm, 3 mm, 5 mm, 7 m, 10 mm, 12 mm, 15 mm, 17 mm, 20 mm, 25 mm, 30 mm, 40 mm, 50 mm, 60 mm, 70 mm, 75 mm, 80 mm, 85 mm, 90 mm, 97 mm, 100 mm, 105 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 270 mm, 300 mm, 350 mm, 400 mm, 450 mm, 500 mm, 550 mm, 600 mm, 700 mm, or 1 m. Alternatively, the device may have a maximum dimension greater than any of the dimension values described herein. In some instances, the device may have a maximum dimension falling within a range between any two of the values described herein.

Any footprint may be provided for the device. The footprint may include a lateral cross-sectional area of the device. The footprint may include an area of a surface that the device would occupy when resting on the surface. In some instances, the device may have a footprint of less than or equal to about 1 cm², 5 cm², 10 cm², 15 cm², 20 cm², 25 cm², 30 cm², 40 cm², 50 cm², 60 cm², 70 cm², 80 cm², 90 cm², 100 cm², 120 cm², 150 cm², 200 cm², 250 cm², 300 cm², 350 cm, 400 cm², 500 cm², 600 cm², 700 cm², 800 cm², 900 cm², 1000 cm², 1200 cm², 1500 cm², 1700 cm², or 2000 cm². The device may have a footprint greater than or equal to any of the values described herein. The device may have a footprint falling into a range between any two of the values described herein.

The device may have any volume. In some instances, the battery may have a volume of less than about 1 cm³, 5 cm³, 10 cm³, 15 cm³, 20 cm³, 25 cm³, 30 cm³, 40 cm³, 50 cm³, 60 cm³, 70 cm³, 80 cm³, 90 cm³, 100 cm³, 120 cm³, 150 cm³, 200 cm³, 250 cm³, 300 cm³, 350 cm³, 400 cm³, 500 cm³, 600 cm³, 700 cm³, 800 cm³, 900 cm³, 1000 cm³, 1200 cm³, 1500 cm³, 1700 cm³, 2000 cm³, 2200 cm³, 2500 cm³, 3000 cm³, 3500 cm³, 4000 cm³, 4500 cm³, 5000 cm³, 5500 cm³, 6000 cm³, 7000 cm³, 8000 cm³, 9000 cm³, or 10,000 cm³. The device may have a volume greater than any of the volumes described herein. The device may have a volume falling within a range between any two of the values described herein.

The device may have any weight. For example, the device may weigh less than or equal to about 2 kg. The device may weigh less than or equal to about 1 mg, 10 mg, 100 mg, 1 g, 10 g, 100 g, 200 g, 300 g, 400 g, 500 g, 600 g, 700 g, 800 g, 900 g, 1 kg, 1.1 kg, 1.2, kg, 1.3 kg, 1.4 kg, 1.45 kg, 1.5 kg, 1.55 kg, 1.6 kg, 1.65 kg, 1.7 kg, 1.75 kg, 1.8 kg, 1.85 kg, 1.9 kg, 2 kg, 2.1 kg, 2.2 kg, 2.5 kg, 2.7 kg, 3 kg, 3.5 kg, 4 kg, 4.5 kg, 5 kg, 6 kg, 7 kg, 8 kg, 9 kg, or 10 kg. The device may weigh more than any of the values described herein. The device may have a weight falling within a range between any two of the values described herein.

Any of the dimensions or characteristics of the device as described herein may be provided separately or in combination with one another. For example, any of the dimensions, footprints, volumes, and/or weights may be combined with one another and/or with any voltage, current, power, described herein. The device may have any characteristics described herein while being configured to conduct nucleic acid amplification and/or real-time detection of the nucleic acid amplification. The device may be a portable device having any of the dimensions described herein while being able to operate at low voltage power. This may advantageously take full advantage of the device's portability, not only in size but ability to be powered from a wider range of power sources and/or have longer battery life.

The device may be configured to accept any number of samples. For example, the device may include any number of indentations, such as those described elsewhere herein. The device may have any number of indentations as described while having any of the dimensions provided. In one example, the device may have 8 indentations. The device may weigh no more than 0.5 kg, 0.4 kg, 0.3 kg, 0.25 kg, 0.2 kg, 0.15 kg, 0.12 kg, or 0.1 kg per indentation. The device may have a footprint of no more than about 500 cm², 300 cm², 200 cm², 150 cm², 100 cm², 70 cm², 60 cm², 50 cm², 40 cm², 30 cm², 20 cm², 10 cm², 5 cm², 1 cm², 100 mm², 10 mm², or 1 mm² per indentation.

The device may be configured to operate using less than or equal to about 25 W, 20 W, 17 W, 15 W, 14 W, 13 W, 12 W, 11 W, 10 W, 9 W, 8 W, 7 W, 6 W, 5 W, 4 W, 3 W, 2 W, 1 W, 500 mW, 100 mW, 50 mW, 10 mW, 5 mW, or 1 mW per indentation.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. An apparatus for processing a sample within a device, said apparatus comprising:
a lid configured to rotate about at least one axis of rotation when said lid is moving between an open position and a closed position; and
a heater configured to translate along a plane without rotating when said lid is moving between the open position and the closed position, wherein said translation is driven by rotation of the lid, and wherein said heater is configured to cover the sample when the lid is in the closed position, and wherein said heater is configured to not cover the sample when the lid is in the open position.

2. The apparatus of claim 1, wherein the device is a thermal cycler.

3. The apparatus of claim 2, wherein the thermal cycler is useful for performing real-time nucleic acid amplification, and said thermal cycler comprises a detector configured to detect a signal from the sample while the nucleic acid amplification is in progress, wherein the signal is related to the amount of amplified nucleic acid present in the sample.

4. The apparatus of claim 1, wherein the sample is contained within a sample container.

5. (canceled)

6. The apparatus of claim 4, wherein said sample container has a container top, and wherein the heater is configured to contact the container top of the sample container when the lid is in the closed position.

7. The apparatus of claim 1, wherein said translation is mechanically driven by the rotation of the lid.

8. The apparatus of claim 1, wherein the lid is coupled to a slider pin that is positioned along a track, wherein movement of the lid causes the slider pin to slide along the track.

9.-11. (canceled)

12. The apparatus of claim 1, further comprising a wire connected to the heater, wherein said translation of the heater causes the wire to change length.

13.-15. (canceled)

16. The apparatus of claim 1, further comprising (i) a processor configured to generate instructions to said heater to control temperature of said heater, and (ii) a housing configured to enclose the sample and the processor.

17. (canceled)

18. The apparatus of claim 16, wherein the heater is within the housing when the lid is in the open position.

19. (canceled)

20. (canceled)

21. An apparatus for processing a sample within a device, said apparatus comprising:
a lid configured to move between an open position and a closed position; and
a heater configured to be separable from the lid and to move along a different path from the lid when said lid is moving between the open position and the closed position, wherein movement of said heater is mechanically driven by movement of the lid, and wherein said heater is configured to cover the sample when the lid is in the closed position, and wherein said heater is configured to not cover the sample when the lid is in the open position.

22. The apparatus of claim 21, wherein the device is a thermal cycler.

23. The apparatus of claim 22, wherein the thermal cycler is useful for performing real-time nucleic acid amplification, and said thermal cycler comprises a detector configured to detect a signal from the sample while the nucleic acid amplification is in progress, wherein the signal is related to the amount of amplified nucleic acid present in the sample.

24. The apparatus of claim 21, wherein the sample is contained within a sample container.

25. (canceled)

26. The apparatus of claim 24, wherein said sample container has a container top, and wherein the heater is configured to contact the container top of the sample container when the lid is in the closed position.

27. The apparatus of claim 21, wherein the lid is coupled to a slider pin that is positioned along a track, wherein movement of the lid causes the slider pin to slide along the track.

28.-30. (canceled)

31. The apparatus of claim 21, further comprising a wire connected to the heater, wherein said movement of the heater causes the wire to change length.

32.-34. (canceled)

35. The apparatus of claim 21, further comprising (i) a processor configured to generate instructions to said heater and to control temperature of said heater, and (ii) a housing configured to enclose the sample and the processor.

36. (canceled)

37. The apparatus of claim 35, wherein the heater is within the housing when the lid is in the open position.

38. (canceled)

39. (canceled)

40. An apparatus for processing a sample within a device, said apparatus comprising:
a lid configured to move between an open position and a closed position; and
a heater configured to move when said lid is moving between the open position and the closed position, and wherein said heater is configured to (1) cover the sample and (2) contact the lid via a force inducing component that exerts a force on the heater toward the sample when the lid is in the closed position, and wherein said heater is configured to (1) not cover the sample and (2) not contact the lid via the force inducing component when the lid is in the open position.

41. The apparatus of claim 40, wherein the heater is configured to move along a different path from the lid.

42. The apparatus of claim 40, wherein movement of said heater is mechanically driven by movement of the lid.

43. The apparatus of claim 40, wherein the force inducing component is a spring.

44. (canceled)

45. (canceled)

46. The apparatus of claim 40, wherein the device is a thermal cycler.

47. The apparatus of claim 46, wherein the thermal cycler is useful for performing real-time nucleic acid amplification, and said thermal cycler comprises a detector configured to detect a signal from the sample while the nucleic acid amplification is in progress, wherein the signal is related to the amount of amplified nucleic acid present in the sample.

48. The apparatus of claim 40, wherein the sample is contained within a sample container.

49. (canceled)

50. The apparatus of claim 48, wherein said sample container has a container top, and wherein the heater is configured to contact the container top of the sample container when the lid is in the closed position.

51. (canceled)

52. The apparatus of claim 40, further comprising (i) a processor configured to generate instructions to said heater to control temperature of said heater, and (ii) a housing configured to enclose the sample and the processor.

53. (canceled)

54. The apparatus of claim 52, wherein the heater is within the housing when the lid is in the open position.

55.-70. (canceled)
